(19) United States
(12) Patent Application Publication
(10) Pub. No.: US 2003/0104977 A1
(43) Pub. Date: Jun. 5, 2003

(54) METHODS FOR INDUCING ANGIOGENESIS USING MORPHOGENIC PROTEINS AND STIMULATORY FACTORS

(76) Inventors: UGO RIPAMONTI, SANDTON (ZA); LENTSHA NATHANIEL RAMOSHEBI, JOHANNESBURG (ZA)

Correspondence Address: FISH & NEAVE, 1251 AVENUE OF THE AMERICAS, 50TH FLOOR, NEW YORK, NY, 10020-1105, US

(21) Appl. No.: 09/540,466

(22) Filed: Mar. 31, 2000

(51) Int. Cl. A61K 38/17

(52) U.S. Cl. 514/2

(57) ABSTRACT

The present invention provides a method for inducing angiogenesis at a target locus in a mammal using morphogenic proteins. In addition, this invention also features a method for improving the angiogenic capability of a morphogenic protein at a target locus in a mammal. In this method, the morphogenic protein is capable of inducing angiogenesis when accessible to a progenitor cell in the mammal, and the morphogenic protein stimulatory factor enhances that capability. The morphogenic protein and morphogenic protein stimulatory factor can be administered simultaneously to the target locus. Alternatively, the two components are administered separately, in any order.

### BACKGROUND OF THE INVENTION

Hemovascular development is a process that involves vasculogenesis, the de novo formation of blood vessels through the aggregation of endothelial cells derived from mesenchyme, and angiogenesis, the growth of new blood vessels from a pre-existing vascular network (Zimrin and Maciag, J. Clin. Invest., 97, p. 1395 (1996); Yancopoulos et al., Cell, 93, pp. 661-664 (1998); Isner and Asahara, J. Clin. Invest., 103, pp.1231-1236 (1999)). Vasculogenesis is normally involved in embryonic development whereas angiogenesis, which also plays a role in the development of the embryo, is of central importance in various physiological and pathological processes in the adult (Folkman, Ann. N.Y. Acad. Sci., 401, pp. 212-227 (1982); Folkman and Klagsbrun, Science, 235, pp. 442-447 (1987); Bussolino et al., Trends Biochem. Sci., 22, pp. 251-256 (1997); Glowacki, Clin. Orthop., 355, pp. S82-S89 (1998); Gerber et al., Nat. Med., 5, pp.623-628 (1999)).

Angiogenesis is a morphogenetic process which plays an important role in the creation of the vascular system during remodeling of adult tissue and in disease. Because of its vital role, angiogenesis must be properly regulated. An equilibrium between angiogenic and anti-angiogenic factors is required for proper angiogenesis. Improper angiogenesis may result in either excessive or inadequate blood vessel growth. For example, excessive vascularization results in rheumatoid arthritis, tumor growth, tumor metastazation and diabetic retinopathy. Inadequate vascularization on the other hand may result in strokes, ischemia and heart attacks including myocardial infarction.

Various physiological processes and pathophysiologies require angiogenesis. These include reproduction, wound healing, organ transplantation, bone repair, ischemic heart disease and ischemic peripheral vascular disease.

Angiogenesis plays a critical role in wound healing. Newly formed capillaries serve as a means to transport cells, nutrients and debris to and from the wound. Angiogenesis is also involved in accelerating healing of inflammatory diseases such as ulcers. Similarly, angiogenesis plays a role in organ transplantation. Vascularization is essential for the functioning of a newly transplanted organ. Angiogenesis allows blood flow into the newly transplanted organ thus providing nutrients for its maintenance.

Angiogenesis also plays a vital role during tissue formation. In order for a specific tissue to form, there is a need for proper vascular invasion of that tissue. For example, during bone formation, in the absence of vascular invasion, only cartilage is formed. If, however, there is vascular invasion, then bone formation is observed.

Myocardial disorders such as myocardial hypertrophy or occlusive coronary artery disease result in myocardial ischemia. These pathologies necessitate an improvement in the vascular supply to the myocardium in order to protect the heart from ischemic damage. Myocardial infarction results in severe tissue damage and necrosis. Angiogenesis functions to remove cellular debris and to provide the heart with the necessary supply of oxygen. There is, therefore, a need to provide methods for enhancing angiogenesis in a mammal.

Several angiogenic factors have been isolated, purified and characterized (Folkman and Klagsburn, Science, 235, pp. 442-447 (1987); Zagzag, Am. J. Pathol., 146, pp. 293-309 (1995); Alini et al., Dev. Biol. 176, pp. 124-133 (1996). For example, fibroblast growth factor (FGF), transforming growth factor-α (TGF-α), transforming growth factory-β (TGF-β) and other related peptides have been identified as having angiogenic activity. However, to date, the angiogenic factors have proven inadequate for the treatment of the pathophysiologies described above. Therefore, new agents and methods of inducing angiogenesis are needed.

The Transforming Growth Factor-Beta (“TGF-β”) superfamily represents a large number of evolutionarily conserved morphogenic proteins with diverse activities in growth, differentiation, tissue morphogenesis and repair. This superfamily includes osteogenic proteins (“OPs”) and bone morphogenic proteins (“BMPs”). OPs and BMPs share a highly conserved, bioactive cysteine-rich domain near their C-termini and have a propensity to form homo- and hetero-dimers.

Many morphogenic proteins belonging to the BMP family have been described. Some were isolated using purification techniques on the basis of osteogenic activity. Others were identified and cloned by virtue of DNA sequence homologies within conserved regions that are common to the BMP family. These homologs are referred to as consecutively numbered BMPs whether or not they have demonstrable osteogenic activity. While several of the earliest members of the BMP family were identified by virtue of their ability to induce new cartilage and bone, a number of other BMPs have different or additional tissue-inductive capabilities. Other BMPs have been reported to induce other tissues. For example, a BMP-like member of the TGF-β superfamily, GDF-5 reportedly has some angiogenic activity. BMP-2, which is a member of the TGF-β superfamily family, however, does not (Yamashita et al., Exp. Cell. Res., 235, pp. 218-226 (1997)). In addition, BMP-12 and BMP-13 (identified by DNA sequence homology) reportedly induce tendon/ligament-like tissue formation in vivo (WO 95/16035). Several BMPs, including some of those originally isolated on the basis of their osteogenic activity, can induce neuron proliferation and promote axon regeneration (WO 95/05846; Liem et al., Cell, 82, pp. 969-79 (1995)). Thus, it appears that BMPs may have a variety of potential tissue-inductive capabilities whose final expression depends on a complex set of developmental and environmental cues.

The availability of large amounts of purified and highly active morphogenic proteins would revolutionize procedures generally involving vascular tissue regeneration. Many of the mammalian OP- and BMP-encoding genes are now cloned and may be recombinantly expressed as active homo- and heterodimeric proteins in a variety of host systems, including bacteria. The ability to recombinantly produce active forms of morphogenic proteins such as OPs and BMPs, including variants and mutants with increased bioactivities (see below), make potential therapeutic treatments using morphogenic proteins feasible.

Given the potential therapeutic uses for morphogenic proteins in inducing angiogenesis, there is a need for highly active forms of morphogenic proteins. It would thus be desirable to increase the angiogenic properties of morphogenic proteins. With increased angiogenic activity, treatment with a morphogenic protein, could induce angiogenesis more rapidly, or angiogenic induction could be achieved using reduced morphogenic protein concentrations.

### SUMMARY OF THE INVENTION

This invention is based on the discovery that morphogenic proteins possess angiogenic activity and that the angiogenic inductive ability of a morphogenic protein can be enhanced by a morphogenic protein stimulatory factor (MPSF).

Accordingly, this invention features a method for inducing angiogenesis at a target locus in a mammal using morphogenic proteins. In addition, this invention also features a method for improving the angiogenic capability of a morphogenic protein at a target locus in a mammal. In this method, the morphogenic protein is capable of inducing angiogenesis when accessible to a progenitor cell in the mammal, and the morphogenic protein stimulatory factor enhances that capability. The morphogenic protein and MPSF can be administered simultaneously to the target locus. Alternatively, the two components are administered separately, in any order.

The morphogenic protein may comprise a pair of subunits disulfide-bonded to produce a dimeric species, wherein at least one of the subunits comprises a polypeptide belonging to the BMP protein family. For instance, the morphogenic protein may comprise an amino acid sequence sufficiently duplicative of the amino acid sequence of a reference BMP such as BMP-2, BMP-3, BMP-4, BMP-5, BMP-6, BMP-7 (OP-1), BMP-8, BMP-9, BMP-10, BMP-11, BMP-12, BMP-13, BMP-14, BMP-15, COP-5, COP-7, such that it has morphogenic activity similar to that of the reference BMP. In one preferred embodiment, the morphogenic protein is a homo- or heterodimer comprising a BMP-7 (OP-1) subunit. Alternatively, the morphogenic protein may comprise a monomeric species. However, when the morphogenic protein is used in the absence of a morphogenic protein stimulatory factor, the morphogenic protein may not be BMP-2 or GDF-5.

The morphogenic protein used in the method of this invention is capable of inducing angiogenesis. For instance, it may be capable of inducing a progenitor cell to form vascular tissue. The method of this invention thus can be used to induce vascular tissue regeneration leading to repair at a tissue defect site.

Morphogenic protein stimulatory factors useful in this invention include but are not limited to hormones, cytokines and growth factors. The MPSF used in the methods of this invention is capable of inducing the angiogenic activity of the morphogenic protein used in this invention.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions will control. The materials, methods and examples are illustrative only and not intended to be limiting.

Other features and advantages of the invention will be apparent from the following drawings, detailed description and the claims.

### BRIEF DESCRIPTION OF THE DRAWINGS

FIG. 1. Illustration of the representative grades used to evaluate the macroscopic vascular reactions in chick chorioallantoic membranes (CAMs) photographed 5 days after the application of Affigel® Blue Gel agarose beads soaked in BSA (500 ng), pTGF-β1 (20 ng), bFGF (500 ng), hOP-1 (100 ng and 1000 ng), hOP-1/bFGF(100/100 ng) or hOp-1/pTGF-β1 (100/5 and 100/20 ng). (A) No response: No change in the distribution of blood vessels in the surrounding CAM and about the application site. (B) Questionable response: blood vessels radiate from the surrounding CAM with more directionality toward the application site. (C) Positive response: blood vessels from the surrounding CAM converge in a spoke-like fashion about the application site. BSA=bovine serum albumin; pTGF-β1=platelet-derived transforming growth factor-β1; bFGF=basic fibroblast growth factor; hOP-1=human osteogenic protein-1. Bars, 1 mm.

FIG. 2. The relative chick chorioallantoic membrane (CAM) thickness ratios in response to the application of Affigel® Blue Gel agarose beads soaked in BSA (500 ng), pTGF-β1 (20 ng), bFGF(500 ng), hOP-1 (100 ng and 1000 ng), hOP-1/bFGF(100/100 ng) or hOP-1/pTGF-β1 (100/5 and 100/20 ng). BSA=bovine serum albumin; pTGF-β1=platelet-derived transforming growth factor-β1; bFGF=basic fibroblast growth factor; hOP-1=human osteogenic protein-1. Values=means±SD, N=5 for all sample groups, P<0.05 by ANOVA.

FIG. 3. Cross section of a typical control-treated chick chorioallantoic membrane (CAM) following exposure to 500 ng of bovine serum albumin (BSA) for 5 days. The area in the vicinity of the beads shows normal structures with thin ectodermal (ec) and endodermal (en) epithelia enclosing the mesodermal (me) stroma. The original positions of some gel beads (g) are distinguishable by indentations in the ectodermal surface of the CAM. The mesoderm consists primarily of sparse and loosely arranged fibroblasts in wide intercellular spaces. Occasional large blood vessels (bv) with nucleated erythrocytes are observed in the mesoderm. The ectoderm exhibits normal development of the intradermal capillaries (iec). Blue staining collagen fibers are sparsely distributed in some regions within the mesoderm. Vestiges of gelatin (gl) remain between the beads and in the regions between the beads and the stratified ectoderm. Scale bar=50 μm.

FIG. 4. Histological response of chick chorioallantoic membrane (CAM) after the application of 20 ng pTGF-β1. There is a distinct thickening of the mesoderm (me) and extensive stratification of the endoderm (en). A widespread proliferation of capillaries (ca) is observed throughout the mesoderm. A discrete accumulation and condensation of the fibrous connective tissue (ct), which is mainly localized in the endodermal portion of the mesoderm, accompanies the increase in the number of capillaries. Blue staining collagen fibers are densely spread in the condensed fibrous tissue within the mesoderm in the locality of the reaction center. Sloughing of the endodermal cells (arrowhead) is observed. Scale bar=100 μm.

FIG. 5. Histological response of chick chorioallantoic membrane (CAM) after exposure to 500 ng of bFGF. There is a distinct thickening of the mesoderm (me) and extensive stratification of both the ectoderm (ec) and endoderm (en). Dense accumulations of fibroblast-rich connective tissue (ct) are localized in areas close to both the ectodermal and the endodermal portions of the mesoderm. Capillaries (ca), as well as a large number of blue-staining collagen fibers, are spread widely throughout the reactive mesoderm. Clusters of cells (cd) with a similar appearance to the stratified ectoderm are embedded within the mesoderm. Blue staining collagen fibers are densely spread in the condensed fibrous tissue within the mesoderm in the locality of the reaction centers and finely spread in the central portion of the mesoderm. Remnants of gelatin (gl) are located between the beads and in the vicinity of the ectoderm. Scale bar=100 μm.

FIG. 6. Histological effects induced by exposure of the chick chorioallantoic membrane (CAM) to hOP-1. (A) 100 ng of hOP-1 induced the development of multiple distended blood vessels (bv), some with nucleated erythrocytes in the lumen, in the loosely arranged mesoderm (me). Increased numbers of capillaries (ca) and a defined fibrous connective tissue (ct) aggregation, including blue staining collagen fibrils, are present within the ectodermal section of the mesoderm. Both the ectoderm (ec) and endoderm (en) are transformed into multilayered epithelia. Sloughing of the ectodermal cells (arrowheads) is clearly evident. Scale bar=50 μm. (B) 1000 ng of hOP-1 induced an accumulation of numerous capillaries (ca) and connective tissue fibers (ct) in the ectodermal segment of the highly expanded mesoderm (me). The ectoderm (ec) is transformed into a multilayered squamous epithelium free of blood vessels. The formerly intraectodermal capillaries are now located underneath the stratified epithelium of the ectoderm to form subepithelial capillaries (sec). The cells of the endoderm (en) are arranged into a multilayered structure. Hydropic and necrotic cells are visible in the clusters of cells (cd) that are morphologically similar to the stratified ectoderm embedded in the thickened mesoderm. Scale bar=50 μm.

FIG. 7. Histological reaction of a chick chorioallantoic membrane (CAM) after the application of a combination of hOP-1/bFGF (100/100 ng). Numerous distended blood vessels (bv) and capillaries (ca) with nucleated erythroytes are widely distributed within the oedematous mesoderm (me). The fibrous connective tissue (ct), consisting of blue staining collagen fibers, is very dense and widely distributed throughout the thickness of the reactive mesoderm. The endoderm (en) and the ectoderm (ec) (not in this section) thickened by stratification. Scale bar=50 μm.

FIG. 8. Chick chorioallantoic membrane (CAM) response following exposure to hOP-1/pTGF-β1. (A) hOP-1/pTGF-β1 (100/5 ng): there is a very marked thickening of all the three layers of the CAM. The multilayered endoderm (en) exhibits a villi-like pattern. Widespread capillaries (ca) and fibrous tissue (ct) are located over the entire reactive mesoderm (me) containing numerous distended blood vessels (bv). Blue staining collagen fibers are densely spread in the condensed fibrous tissue within the mesoderm in the locality of the areas adjacent to the ecto- and endoderm and finely spread in the central portion of the mesoderm. Clusters of cells (cd) with a similar appearance to the stratified ectoderm are embedded within the mesoderm. Sloughing of the endoderm (arrowheads) is clearly visible. Scale bar=50 μm. (B) hOP-1/pTGF-β1 (100/20 ng): There is extensive fibrous tissue (ct) condensation and prominently high number of capillaries (ca). Evidence of bead (g) encapsulation is clearly noticeable. The dense connective tissue fibers including the blue-staining collagen, are aligned in the region skirting the zone of encapsulated beads. The multilayered endoderm (en) is villi-like and the thickened ectoderm is vessel-free. Sloughing of the endoderm (arrowhead) is clearly visible. Scale bar =100 μm.

FIG. 9. Photomicrographic evaluation of the chick chorioallantoic membrane (CAM) angiogenic response to the application of pTGF-β1, bFGF, hOP-1, hOP-1/bFGF or hOP-1/pTGF-β1 using Affigel® Blue Gel agarose beads. N=8 for all sample groups, P<0.05 by ANOVA. BSA=bovine serum albumin; pTGF-β1=platelet-derived transforming growth factor-β1; bFGF=basic fibroblast growth factor; hOP-1 =human osteogenic protein-1.

FIG. 10. Qualitative ranking of chick chorioallantoic membrane (CAM) angiogenic responses to the application of pTGF-β1, bFGF, hOP-1, hOP-1/bFGF or hOP-1/pTGF-β1 using Affi-Gel® Blue Gel agarose beads. Quantities are in nanograms. N=5 for all sample groups. BSA=bovine serum albumin; pTGF-β1=platelet-derived transforming growth factor-β1; bFGF=basic fibroblast growth factor; hOP-1 =human osteogenic protein-1.

### DETAILED DESCRIPTION OF THE INVENTION

In order that the invention herein described may be fully understood, the following detailed description is set forth.

The term “biocompatible” refers to a material that does not elicit detrimental effects associated with the body's various protective systems, such as cell and humoral-associated immune responses, e.g., inflammatory responses and foreign body fibrotic responses. This term also implies that no specific undesirable effects, cytotoxic or systemic, are caused by the material when it is implanted into the patient.

The term “BMP” refers to a protein belonging to the BMP family of the TGF-β superfamily of proteins defined on the basis of DNA and amino acid sequence homology. According to this invention, a protein belongs to the BMP family when it has at least 70% (e.g., at least 80% or even 85%) amino acid sequence homology with a known BMP family member within the conserved C-terminal cysteine-rich domain that characterizes the BMP family. Members of the BMP family may have less than 70% DNA or amino acid sequence homology overall.

The term “morphogenic protein” refers to a protein having morphogenic activity. For instance, this protein is capable of inducing progenitor cells to proliferate and/or to initiate differentiation pathways that lead to the formation of cartilage, bone, tendon, ligament, vascular, neural or other types of tissue, depending on local environmental cues. Thus, morphogenic proteins useful in this invention may behave differently in different surroundings. A morphogenic protein of this invention may comprise at least one polypeptide belonging to the BMP family.

The term “osteogenic protein” refers to a morphogenic protein that is capable of inducing a progenitor cell to form cartilage and/or bone. The bone may be intramembranous bone or endochondral bone. Most osteogenic proteins are members of the BMP family and are thus also BMPs. However, the converse may not be true. According to this invention, a BMP identified by sequence homology must have demonstrable osteogenic or chondrogenic activity in a functional bioassay to be an osteogenic protein.

The term “morphogenic protein stimulatory factor (MPSF)” refers to a factor that is capable of stimulating the ability of a morphogenic protein to induce tissue formation from a progenitor cell. The MPSF may have a direct or indirect effect on enhancing morphogenic protein inducing activity. For example, the MPSF may increase the bioactivity of another MPSF. Agents that increase MPSF bioactivity include, for example, those that increase the synthesis, half-life, reactivity with other biomolecules such as binding proteins and receptors, or the bioavailability of the MPSF.

The terms “morphogenic activity,” “inducing activity” and “tissue inductive activity” alternatively refer to the ability of an agent to stimulate a target cell to undergo one or more cell divisions (proliferation) that may optionally lead to cell differentiation. Such target cells are referred to generically herein as progenitor cells. Cell proliferation is typically characterized by changes in cell cycle regulation and may be detected by a number of means which include measuring DNA synthetic or cellular growth rates. Early stages of cell differentiation are typically characterized by changes in gene expression patterns relative to those of the progenitor cell; such changes may be indicative of a commitment towards a particular cell fate or cell type. Later stages of cell differentiation may be characterized by changes in gene expression patterns, cell physiology and morphology. Any reproducible change in gene expression, cell physiology or morphology may be used to assess the initiation and extent of cell differentiation induced by a morphogenic protein.

The terms “angiogenesis” and “angiogenic activity” alternatively refer to the ability of an agent to stimulate the formation of blood vessels and associated cells (including endothelial, perivascular, mesenchymal, and smooth muscle cells) and blood vessel associated basement membrane. This includes, for example of new capillary blood vessels from existent microvessels by sprouting, i.e., cellular outgrowth.

The term “synergistic interaction” refers to an interaction in which the combined effect of two or more agents is greater than the algebraic sum of their individual effects.

Provided below are detailed descriptions of suitable morphogenic proteins and morphogenic protein stimulatory factors useful in the methods of this invention. Specifically, the examples provide models for demonstrating the utility of the morphogenic proteins in inducing angiogenesis.

Morphogenic Proteins

The morphogenic proteins used in the methods of this invention are capable of stimulating a progenitor cell to undergo cell division and/or differentiation. They may belong to the TGF-β protein superfamily, and include, but are not limited to, OP-1, OP-2, OP-3, BMP-2, BMP-3, BMP-3b, BMP-4, BMP-5, BMP-6, BMP-9, BMP-10, BMP-11, BMP-12, BMP-13, BMP-14, BMP-15, GDF-1, GDF-2, GDF-3, GDF-5, GDF-6, GDF-7, GDF-8, GDF-9, GDF-10, GDF-11, GDF-12, DPP, Vg-1, Vgr-1, 60A protein, NODAL, UNIVIN, SCREW, ADMP, and NEURAL. However, when the morphogenic protein is used in the absence of a morphogenic protein stimulatory factor, the morphogenic protein may not be BMP-2 or GDF-5.

In a preferred embodiment, the morphogenic protein comprises an amino acid sequence selected from the group consisting of BMP-3, BMP-4, BMP-5, BMP-6, OP-1 (BMP-7), BMP-8, BMP-9, BMP-10, BMP-11, BMP-12, BMP-13, BMP-14, BMP-15, COP-5, COP-7 and an amino acid sequence variant thereof. In a more preferred embodiment, the morphogenic protein comprises an amino acid sequence selected from the group consisting of OP-1, BMP-5, BMP-6, BMP-8, GDF-6, GDF-7 and amino acid sequence variants thereof. In a most preferred embodiment, the morphogenic protein is OP-1.

One of the preferred morphogenic proteins that is useful in this invention is OP-1. Nucleotide and amino acid sequences for hOP-1 are provided in SEQ ID NOs: 1 and 2, respectively. For ease of description, hOP-1 is recited as a representative morphogenic protein. It will be appreciated by the ordinarily skilled artisan that OP-1 is merely representative of a family of morphogens.

Other useful morphogenic proteins include polypeptides having at least 70% (e.g., at least 80% or even 85%) sequence homology with a known morphogenic protein, particularly with a known BMP within the conserved C-terminal cysteine-rich domain that characterizes the BMP protein family. These morphogenic proteins include biologically active variants of any known morphogenic protein, including variants containing conservative amino acid changes. For instance, useful morphogenic proteins include those containing sequences that share at least 70% amino acid sequence homology with the C-terminal seven-cysteine domain of hOP-1, which domain corresponds to the C-terminal 102-106 amino acid residues of SEQ ID NO: 2. The C-terminal 102 amino acid residues corresponds to residues 330-431 of SEQ ID NO: 2. In one embodiment of this invention, the morphogenic protein used consists of a pair of subunits disulfide-bonded to produce a dimer, wherein at least one of the subunits comprises a recombinant polypeptide belonging to the BMP family. In another embodiment of this invention, the morphogenic protein used consists of a monomeric polypeptide belonging to the BMP family.

As used herein, “amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. Certain particularly preferred morphogenic polypeptides share at least 60% (e.g., at least 65%) amino acid sequence identity with the C-terminal seven-cysteine domain of human OP-1.

As used herein, “conservative substitutions” are residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure, 5, pp. 345-362 (1978 & Supp.). Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative variant” or “conservative variation” also includes the use of a substituting amino acid residue in place of an amino acid residue in a given parent amino acid sequence, where antibodies specific for the parent sequence are also specific for, i.e., “cross-react” or “immuno-react” with, the resulting substituted polypeptide sequence.

Amino acid sequence homology can be determined by methods well known in the art. For instance, to determine the percent homology of a candidate amino acid sequence to the sequence of the seven-cysteine domain, the two sequences are first aligned. The alignment can be made with, e.g., the dynamic programming algorithm described in Needleman et al., J. Mol. Biol., 48, p. 443 (1970), and the Align Program, a commercial software package produced by DNAstar, Inc. The teachings by both sources are incorporated by reference herein. An initial alignment can be refined by comparison to a multi-sequence alignment of a family of related proteins. Once the alignment is made and refined, a percent homology score is calculated. The aligned amino acid residues of the two sequences are compared sequentially for their similarity to each other. Similarity factors include similar size, shape and electrical charge. One particularly preferred method of determining amino acid similarities is the PAM250 matrix described in Dayhoff et al., supra. A similarity score is first calculated as the sum of the aligned pairwise amino acid similarity scores. Insertions and deletions are ignored for the purposes of percent homology and identity. Accordingly, gap penalties are not used in this calculation. The raw score is then normalized by dividing it by the geometric mean of the scores of the candidate sequence and the seven-cysteine domain. The geometric mean is the square root of the product of these scores. The normalized raw score is the percent homology.

Morphogenic proteins useful herein include any known naturally occurring native proteins, including allelic, phylogenetic counterparts and other variants thereof. These variants include forms having varying glycosylation patterns, varying N-termini, and active truncated or mutated forms of a native protein. Useful morphogenic proteins also include those that are biosynthetically produced (e.g., “muteins” or “mutant proteins”) and those that are new, morphogenically active members of the general morphogenic family of proteins. Particularly useful sequences include those comprising the C-terminal 96 to 102 amino acid residues of: DPP (from Drosophila), Vg-1 (from Xenopus), Vgr-1 (from mouse), the OP1 and OP2 proteins (U.S. Pat. No. 5,011,691), as well as the proteins referred to as BMP-2, BMP-3, BMP-4 (WO 88/00205, U.S. Pat. No. 5,013,649 and WO 91/18098), BMP-5 and BMP-6 (WO 90/11366), BMP-8 and BMP-9. Other proteins useful in the practice of the invention include active forms of OP1, OP2, OP3, BMP-2, BMP-3, BMP-3b, BMP-4, BMP-5, BMP-6, BMP-9, BMP-10, BMP-11, BMP-12, BMP-13, BMP-14, BMP-15, DPP, Vg-1, Vgr-1, 60A protein, GDF-1, GDF-2, GDF-3, GDF-5, GDF-6, GDF-7, GDF-8, GDF-9, and GDF-10, GDF-11, GDF-12, GDF-13, UNIVIN, NODAL, SCREW, ADMP, and NEURAL. However, when the morphogenic protein is used in the absence of a morphogenic protein stimulatory factor, the morphogenic protein may not be BMP-2 or GDF-5.

Osteogenic proteins useful as morphogenic proteins of this invention include those containing sequences that share greater than 60% identity with the seven-cysteine domain. In other embodiments, useful osteogenic proteins are defined as osteogenically active proteins having any one of the generic sequences defined herein, including OPX (SEQ ID NO: 3) and Generic Sequences 7 (SEQ ID NO: 4), 8 (SEQ ID NO: 5), 9 (SEQ ID NO: 6) and 10 (SEQ ID NO: 7).

Generic Sequence 7 (SEQ ID NO: 4) and Generic Sequence 8 (SEQ ID NO: 5), disclosed below, accommodate the homologies shared among preferred protein family members identified to date, including OP-1, OP-2, OP-3, BMP-2, BMP-3, BMP-4, BMP-5, BMP-6, 60A, DPP, Vg-1, Vgr-1, and GDF-1. The amino acid sequences for these proteins are described herein and/or in the art. The generic sequences include the identical amino acid residues shared by these sequences in the C-terminal six- or seven-cysteine skeletal domains (represented by Generic Sequences 7 and 8, respectively), as well as alternative residues for the variable positions within the sequences. The generic sequences provide an appropriate cysteine skeleton where inter- or intra-molecular disulfide bonds can form. Those sequences contain certain specified amino acids that may influence the tertiary structure of the folded proteins. In addition, the generic sequences allow for an additional cysteine at position 36 (Generic Sequence 7) or position 41 (Generic Sequence 8), thereby encompassing the biologically active sequences of OP-2 and OP-3.

| | |
|---|---|
| GENERIC SEQUENCE 7 | (SEQ ID NO:4) |

```
Leu Xaa Xaa Xaa Phe Xaa Xaa Xaa Gly Trp Xaa Xaa
  1 5 10
Xaa Xaa Xaa Xaa Pro Xaa Xaa Xaa Xaa Ala Xaa Tyr
                15 20
Cys Xaa Gly Xaa Cys Xaa Xaa Pro Xaa Xaa Xaa Xaa
25 30 35
Xaa Xaa Xaa Xaa Asn His Ala Xaa Xaa Xaa Xaa Xaa
                        40 45
Xaa Xaa Xaa Xaa Xaa Xaa Xaa Xaa Xaa Xaa Xaa Xaa
        50 55 60
Cys Cys Xaa Pro Xaa Xaa Xaa Xaa Xaa Xaa Xaa Xaa
                                65 70
Leu Xaa Xaa Xaa Xaa Xaa Xaa Xaa Val Xaa Leu Xaa
              75 80
Xaa Xaa Xaa Xaa Met Xaa Val Xaa Xaa Cys Xaa Cys
85 90 95
Xaa
```

wherein each Xaa is independently defined as follows (“Res.” means “residue”): Xaa at res.2=(Tyr or Lys); Xaa at res.3=(Val or Ile); Xaa at res.4=(Ser, Asp or Glu); Xaa at res.6=(Arg, Gln, Ser, Lys or Ala); Xaa at res.7=(Asp or Glu); Xaa at res.8=(Leu, Val or Ile); Xaa at res.11=(Gln, Leu, Asp, His, Asn or Ser); Xaa at res.12=(Asp, Arg, Asn or Glu); Xaa at res. 13=(Trp or Ser); Xaa at res.14=(Ile or Val); Xaa at res.15=(Ile or Val); Xaa at res.16 (Ala or Ser); Xaa at res.18=(Glu, Gln, Leu, Lys, Pro or Arg); Xaa at res.19=(Gly or Ser); Xaa at res.20=(Tyr or Phe); Xaa at res.21=(Ala, Ser, Asp, Met, His, Gln, Leu or Gly); Xaa at res.23=(Tyr, Asn or Phe); Xaa at res.26=(Glu, His, Tyr, Asp, Gln, Ala or Ser); Xaa at res.28=(Glu, Lys, Asp, Gln or Ala); Xaa at res.30=(Ala, Ser, Pro, Gln, Ile or Asn); Xaa at res.31=(Phe, Leu or Tyr); Xaa at res.33=(Leu, Val or Met); Xaa at res.34=(Asn, Asp, Ala, Thr or Pro); Xaa at res.35=(Ser, Asp, Glu, Leu, Ala or Lys); Xaa at res.36=(Tyr, Cys, His, Ser or Ile); Xaa at res.37=(Met, Phe, Gly or Leu); Xaa at res.38=(Asn, Ser or Lys); Xaa at res.39=(Ala, Ser, Gly or Pro); Xaa at res.40=(Thr, Leu or Ser); Xaa at res.44=(Ile, Val or Thr); Xaa at res.45=(Val, Leu, Met or Ile); Xaa at res.46=(Gln or Arg); Xaa at res.47=(Thr, Ala or Ser); Xaa at res.48=(Leu or Ile); Xaa at res.49=(Val or Met); Xaa at res.50=(His, Asn or Arg); Xaa at res.51=(Phe, Leu, Asn, Ser, Ala or Val); Xaa at res.52=(Ile, Met, Asn, Ala, Val, Gly or Leu); Xaa at res.53=(Asn, Lys, Ala, Glu, Gly or Phe); Xaa at res.54=(Pro, Ser or Val); Xaa at res.55=(Glu, Asp, Asn, Gly, Val, Pro or Lys); Xaa at res.56=(Thr, Ala, Val, Lys, Asp, Tyr, Ser, Gly, Ile or His); Xaa at res.57=(Val, Ala or Ile); Xaa at res.58=(Pro or Asp); Xaa at res.59=(Lys, Leu or Glu); Xaa at res.60=(Pro, Val or Ala); Xaa at res.63=(Ala or Val); Xaa at res.65=(Thr, Ala or Glu); Xaa at res.66=(Gln, Lys, Arg or Glu); Xaa at res.67=(Leu, Met or Val); Xaa at res.68=(Asn, Ser, Asp or Gly); Xaa at res.69=(Ala, Pro or Ser); Xaa at res.70=(Ile, Thr, Val or Leu); Xaa at res.71=(Ser, Ala or Pro); Xaa at res.72=(Val, Leu, Met or Ile); Xaa at res.74=(Tyr or Phe); Xaa at res.75=(Phe, Tyr, Leu or His); Xaa at res.76=(Asp, Asn or Leu); Xaa at res.77=(Asp, Glu, Asn, Arg or Ser); Xaa at res.78=(Ser, Gln, Asn, Tyr or Asp); Xaa at res.79=(Ser, Asn, Asp, Glu or Lys); Xaa at res.80=(Asn, Thr or Lys); Xaa at res.82=(Ile, Val or Asn); Xaa at res.84=(Lys or Arg); Xaa at res.85=(Lys, Asn, Gln, His, Arg or Val); Xaa at res.86=(Tyr, Glu or His); Xaa at res.87=(Arg, Gln, Glu or Pro); Xaa at res.88=(Asn, Glu, Trp or Asp); Xaa at res.90=(Val, Thr, Ala or Ile); Xaa at res.92=(Arg, Lys, Val, Asp, Gln or Glu); Xaa at res.93=(Ala, Gly, Glu or Ser); Xaa at res.95=(Gly or Ala); and Xaa at res.97=(His or Arg).

Generic Sequence 8 (SEQ ID NO: 5) includes all of Generic Sequence 7 and in addition includes the following five amino acid at its N-terminus: Cys Xaa Xaa Xaa Xaa (SEQ ID NO: 8), wherein Xaa at res.2=(Lys, Arg, Ala or Gln); Xaa at res.3=(Lys, Arg or Met); Xaa at res.4=(His, Arg or Gln); and Xaa at res.5=(Glu, Ser, His, Gly, Arg, Pro, Thr, or Tyr). Accordingly, beginning with residue 7, each “Xaa” in Generic Sequence 8 is a specified amino acid as defined as for Generic Sequence 7, with the distinction that each residue number described for Generic Sequence 7 is shifted by five in Generic Sequence 8. For example, “Xaa at res.2=(Tyr or Lys)” in Generic Sequence 7 corresponds to Xaa at res.7 in Generic Sequence 8.

Generic Sequences 9 (SEQ ID NO: 6) and 10 (SEQ ID NO: 7) are composite amino acid sequences of the following proteins: human OP-1 (“hOP-1”), hOP-2, hOP-3, hBMP-2, hBMP-3, hBMP-4, hBMP-5, hBMP-6, hBMP-9, hBMP10, hBMP-11, Drosophila 60A, Xenopus Vg-1, sea urchin UNIVIN, hCDMP-1 (mouse GDF-5 or “mGDF-5”), hCDMP-2 (mGDF-6, hBMP-13), hCDMP-3 (mGDF-7, hBMP-12), mGDF-3, hGDF-1, mGDF-1, chicken DORSALIN, DPP, Drosophila SCREW, mouse NODAL, mGDF-8, hGDF-8, mGDF-9, mGDF-10, hGDF-11, mGDF-11, hBMP-15, and rat BMP3b. Like Generic Sequence 7, Generic Sequence 9 accommodates the C-terminal six-cysteine skeleton and, like Generic Sequence 8, Generic Sequence 10 accommodates the C-terminal seven-cysteine skeleton.

| | |
|---|---|
| GENERIC SEQUENCE 9 | |
| Xaa Xaa Xaa Xaa Xaa Xaa Xaa Xaa Xaa Xaa Xaa Xaa | (SEQ ID NO:6) |
| 1 5 10 | |
| Xaa Xaa Xaa Xaa Pro Xaa Xaa Xaa Xaa Xaa Xaa Xaa Cys Xaa | |
| 15 20 25 | |
| Gly Xaa Cys Xaa Xaa Xaa Xaa Xaa Xaa Xaa Xaa Xaa Xaa Xaa | |
| 30 35 40 | |
| Xaa Xaa Xaa Xaa Xaa Xaa Xaa Xaa Xaa Xaa Xaa Xaa Xaa Xaa | |
| 45 50 | |
| Xaa Xaa Xaa Xaa Xaa Xaa Xaa Cys Xaa Pro Xaa Xaa Xaa Xaa | |
| 55 60 65 | |
| Xaa Xaa Xaa Xaa Leu Xaa Xaa Xaa Xaa Xaa Xaa Xaa Xaa Xaa | |
| 70 75 80 | |
| Xaa Xaa Xaa Xaa Xaa Xaa Xaa Xaa Xaa Xaa Xaa Cys Xaa Cys | |
| 85 90 95 | |
| Xaa | |

wherein each Xaa is independently defined as follows: Xaa at res.1=(Phe, Leu or Glu); Xaa at res.2=(Tyr, Phe, His, Arg, Thr, Lys, Gln, Val or Glu); Xaa at res.3=(Val, Ile, Leu or Asp); Xaa at res.4=(Ser, Asp, Glu, Asn or Phe); Xaa at res.5=(Phe or Glu); Xaa at res.6=(Arg, Gln, Lys, Ser, Glu, Ala or Asn); Xaa at res.7=(Asp, Glu, Leu, Ala or Gln); Xaa at res.8=(Leu, Val, Met, Ile or Phe); Xaa at res.9=(Gly, His or Lys); Xaa at res.10=(Trp or Met); Xaa at res.11=(Gln, Leu, His, Glu, Asn, Asp, Ser or Gly); Xaa at res.12=(Asp, Asn, Ser, Lys, Arg, Glu or His); Xaa at res.13=(Trp or Ser); Xaa at res.14=(Ile or Val); Xaa at res.15=(Ile or Val); Xaa at res.16=(Ala, Ser, Tyr or Trp); Xaa at res.18=(Glu, Lys, Gln, Met, Pro, Leu, Arg, His or Lys); Xaa at res.19=(Gly, Glu, Asp, Lys, Ser, Gln, Arg or Phe); Xaa at res.20=(Tyr or Phe); Xaa at res.21=(Ala, Ser, Gly, Met, Gln, His, Glu, Asp, Leu, Asn, Lys or Thr); Xaa at res.22=(Ala or Pro); Xaa at res.23=(Tyr, Phe, Asn, Ala or Arg); Xaa at res.24=(Tyr, His, Glu, Phe or Arg); Xaa at res.26=(Glu, Asp, Ala, Ser, Tyr, His, Lys, Arg, Gln or Gly); Xaa at res.28=(Glu, Asp, Leu, Val, Lys, Gly, Thr, Ala or Gln); Xaa at res.30=(Ala, Ser, Ile, Asn, Pro, Glu, Asp, Phe, Gln or Leu); Xaa at res.31=(Phe, Tyr, Leu, Asn, Gly or Arg); Xaa at res.32=(Pro, Ser, Ala or Val); Xaa at res.33=(Leu, Met, Glu, Phe or Val); Xaa at res.34=(Asn, Asp, Thr, Gly, Ala, Arg, Leu or Pro); Xaa at res.35=(Ser, Ala, Glu, Asp, Thr, Leu, Lys, Gln or His); Xaa at res.36=(Tyr, His, Cys, Ile, Arg, Asp, Asn, Lys, Ser, Glu or Gly); Xaa at res.37=(Met, Leu, Phe, Val, Gly or Tyr); Xaa at res.38=(Asn, Glu, Thr, Pro, Lys, His, Gly, Met, Val or Arg); Xaa at res.39=(Ala, Ser, Gly, Pro or Phe); Xaa at res.40=(Thr, Ser, Leu, Pro, His or Met); Xaa at res.41=(Asn, Lys, Val, Thr or Gln); Xaa at res.42=(His, Tyr or Lys); Xaa at res.43=(Ala, Thr, Leu or Tyr); Xaa at res.44=(Ile, Thr, Val, Phe, Tyr, Met or Pro); Xaa at res.45=(Val, Leu, Met, Ile or His); Xaa at res.46=(Gln, Arg or Thr); Xaa at res.47=(Thr, Ser, Ala, Asn or His); Xaa at res.48=(Leu, Asn or Ile); Xaa at res.49=(Val, Met, Leu, Pro or Ile); Xaa at res.50=(His, Asn, Arg, Lys, Tyr or Gln); Xaa at res.51=(Phe, Leu, Ser, Asn, Met, Ala, Arg, Glu, Gly or Gln); Xaa at res.52=(Ile, Met, Leu, Val, Lys, Gln, Ala or Tyr); Xaa at res.53=(Asn, Phe, Lys, Glu, Asp, Ala, Gln, Gly, Leu or Val); Xaa at res.54=(Pro, Asn, Ser, Val or Asp); Xaa at res.55=(Glu, Asp, Asn, Lys, Arg, Ser, Gly, Thr, Gln, Pro or His); Xaa at res.56=(Thr, His, Tyr, Ala, Ile, Lys, Asp, Ser, Gly or Arg); Xaa at res.57=(Val, Ile, Thr, Ala, Leu or Ser); Xaa at res.58=(Pro, Gly, Ser, Asp or Ala); Xaa at res.59=(Lys, Leu, Pro, Ala, Ser, Glu, Arg or Gly); Xaa at res.60=(Pro, Ala, Val, Thr or Ser); Xaa at res.61 (Cys, Val or Ser); Xaa at res.63=(Ala, Val or Thr); Xaa at res.65=(Thr, Ala, Glu, Val, Gly, Asp or Tyr); Xaa at res.66=(Gln, Lys, Glu, Arg or Val); Xaa at res.67=(Leu, Met, Thr or Tyr); Xaa at res.68=(Asn, Ser, Gly, Thr, Asp, Glu, Lys or Val); Xaa at res.69=(Ala, Pro, Gly or Ser); Xaa at res.70=(Ile, Thr, Leu or Val); Xaa at res.71=(Ser, Pro, Ala, Thr, Asn or Gly); Xaa at res.72=(Val, Ile, Leu or Met); Xaa at res.74=(Tyr, Phe, Arg, Thr, Tyr or Met); Xaa at res.75=(Phe, Tyr, His, Leu, Ile, Lys, Gln or Val); Xaa at res.76=(Asp, Leu, Asn or Glu); Xaa at res.77=(Asp, Ser, Arg, Asn, Glu, Ala, Lys, Gly or Pro); Xaa at res.78=(Ser, Asn, Asp, Tyr, Ala, Gly, Gln, Met, Glu, Asn or Lys); Xaa at res.79=(Ser, Asn, Glu, Asp, Val, Lys, Gly, Gln or Arg); Xaa at res.80=(Asn, Lys, Thr, Pro, Val, Ile, Arg, Ser or Gln); Xaa at res.81=(Val, Ile, Thr or Ala); Xaa at res.82=(Ile, Asn, Val, Leu, Tyr, Asp or Ala); Xaa at res.83=(Leu, Tyr, Lys or Ile); Xaa at res.84=(Lys, Arg, Asn, Tyr, Phe, Thr, Glu or Gly); Xaa at res.85=(Lys, Arg, His, Gln, Asn, Glu or Val); Xaa at res.86=(Tyr, His, Glu or Ile); Xaa at res.87=(Arg, Glu, Gln, Pro or Lys); Xaa at res.88=(Asn, Asp, Ala, Glu, Gly or Lys); Xaa at res.89=(Met or Ala); Xaa at res.90=(Val, Ile, Ala, Thr, Ser or Lys); Xaa at res.91=(Val or Ala); Xaa at res.92=(Arg, Lys, Gln, Asp, Glu, Val, Ala, Ser or Thr); Xaa at res.93=(Ala, Ser, Glu, Gly, Arg or Thr); Xaa at res.95=(Gly, Ala or Thr); and Xaa at res.97=(His, Arg, Gly, Leu or Ser). Further, after res.53 in rat BMP3b and mGDF-10 there is an Ile; after res.54 in GDF-1 there is a Thr; after res.54 in BMP3 there is a Val; after res.78 in BMP-8 and DORSALIN there is a Gly; after res.37 in hGDF-1 there are Pro, Gly, Gly, and Pro.

Generic Sequence 10 (SEQ ID NO: 7) includes all of Generic Sequence 9 and in addition includes the following five amino acid residues at its N-terminus: Cys Xaa Xaa Xaa Xaa (SEQ ID NO: 9), wherein Xaa at res.2=(Lys, Arg, Gln, Ser, His, Glu, Ala, or Cys); Xaa at res.3=(Lys, Arg, Met, Lys, Thr, Leu, Tyr, or Ala); Xaa at res.4=(His, Gln, Arg, Lys, Thr, Leu, Val, Pro, or Tyr); and Xaa at res.5=(Gln, Thr, His, Arg, Pro, Ser, Ala, Gln, Asn, Tyr, Lys, Asp, or Leu). Accordingly, beginning at res.6, each “Xaa” in Generic Sequence 10 is a specified amino acid defined as for Generic Sequence 9, with the distinction that each residue number described for Generic Sequence 9 is shifted by five in Generic Sequence 10. For example, “Xaa at res.1=(Phe, Leu or Glu)” in Generic Sequence 9 corresponds to Xaa at res.6 in Generic Sequence 10.

As noted above, certain preferred bone morphogenic proteins useful in this invention have greater than 60%, preferably greater than 65%, identity with the C-terminal seven-cysteine domain of hOP-1. These particularly preferred sequences include allelic and phylogenetic variants of the OP-1 and OP-2 proteins, including the Drosophila 60A protein. Accordingly, in certain particularly preferred embodiments, useful proteins include active proteins comprising dimers having the generic amino acid sequence “OPX” (SEQ ID NO: 3), which defines the seven-cysteine skeleton and accommodates the homologies between several identified variants of OP-1 and OP-2. Each Xaa in OPX is independently selected from the residues occurring at the corresponding position in the C-terminal sequence of mouse or human OP-1 or OP-2.

| OPX | |
|---|---|
| Cys Xaa Xaa His Glu Leu Tyr Val Ser Phe Xaa Asp Leu Gly | (SEQ ID NO:3) |
| 1 5 10 | |
| Trp Xaa Asp Trp Xaa Ile Ala Pro Xaa Gly Tyr Xaa Ala Tyr | |
| 15 20 25 | |
| Tyr Cys Glu Gly Glu Cys Xaa Phe Pro Leu Xaa Ser Xaa Met | |
| 30 35 40 | |
| Asn Ala Thr Asn His Ala Ile Xaa Gln Xaa Leu Val His Xaa | |
| 45 50 55 | |
| Xaa Xaa Pro Xaa Xaa Val Pro Lys Xaa Cys Cys Ala Pro Thr | |
| 60 65 70 | |
| Xaa Leu Xaa Ala Xaa Ser Val Leu Tyr Xaa Asp Xaa Ser Xaa | |
| 75 80 | |
| Asn Val Ile Leu Xaa Lys Xaa Arg Asn Met Val Val Xaa Ala | |
| 85 90 95 | |
| Cys Gly Cys His | |
| 100 | |

wherein Xaa at res.2=(Lys or Arg); Xaa at res.3=(Lys or Arg); Xaa at res.11=(Arg or Gln); Xaa at res.16=(Gln or Leu); Xaa at res.19=(Ile or Val); Xaa at res.23=(Glu or Gln); Xaa at res.26=(Ala or Ser); Xaa at res.35=(Ala or Ser); Xaa at res.39=(Asn or Asp); Xaa at res.41=(Tyr or Cys); Xaa at res.50=(Val or Leu); Xaa at res.52=(Ser or Thr); Xaa at res.56=(Phe or Leu); Xaa at res.57=(Ile or Met); Xaa at res.58=(Asn or Lys); Xaa at res.60=(Glu, Asp or Asn); Xaa at res.61=(Thr, Ala or Val); Xaa at res.65=(Pro or Ala); Xaa at res.71=(Gln or Lys); Xaa at res.73=(Asn or Ser); Xaa at res.75=(Ile or Thr); Xaa at res.80=(Phe or Tyr); Xaa at res.82=(Asp or Ser); Xaa at res.84=(Ser or Asn); Xaa at res.89=(Lys or Arg); Xaa at res.91=(Tyr or His); and Xaa at res.97=(Arg or Lys).

In another embodiment, the morphogenic proteins used in the methods of this invention comprise species of the generic amino acid sequence

| | |
|---|---|
| 1 10 20 30 | (SEQ ID NO:10) |
| CXXXXLXVXFXDXGWXXWXXXPXGXXAXYCXGXCXX | |
| 40 50 60 70 | |
| PXXXXXXXXNHAXXQXXVXXXNXXXXPXXCCXPXXX | |
| 80 90 100 | |
| XXXXXLXXXXXXXVXLXXYXXMXVXXCXCX | |

or residues 6-102 of SEQ ID NO: 10, where the letters indicate the amino acid residues of standard single letter code, and the Xs represent any amino acid residues. Cysteine residues are highlighted.

Preferred amino acid sequences within the foregoing generic sequence (SEQ ID NO: 10) are:

```
1 10 20 30 40 50
        LYVDFRDVGWNDWIVAPPGYHAFYCHGECPFPLADHLNSTNHAIV
          K S S L QE VIS E FD Y E A AY MPESMKAS VI
          F E K I DN L N S Q ITK F P TL
                  A S K
                60 70 80 90 100
QTLVNSVNPGKIPKACCVPTELSATSMLYLDENENVVLKNYQDMVVEGCGCR
  SI HAI SEQV EP EQMNSLAI FFNDQDK I RK EE T DA H H
        RF T S K DPV V Y N S H RN RS
          N S K P E
and
1 10 20 30 40 50
  CKRHPLYVDFRDVGWNDWIVAPPGYHAFYCHGECPFPLADHLNSTNHAIV
    RRRS K S S L QE VIS E FD Y E A AY MPESMKAS VI
        KE F E K I DN L N S Q ITK F P TL
Q A S K
                60 70 80 90 100
QTLVNSVNPGKIPKACCVPTELSAISMLYLDENENVVLKNYQDMVVEGCGCR
  SI HAI SEQV EP EQMNSLAI FFNDQDK I RK EE T DA H H
        RF T S K DPV V Y N S H RN RS
          N S K P E
```

wherein each of the amino acids arranged vertically at each position in the sequence may be used alternatively in various combinations (SEQ ID NO: 10). These generic sequences have 6 or 7 cysteine residues where inter- or intra-molecular disulfide bonds can form. These sequences also contain other critical amino acids that influence the tertiary structure of the proteins.

In still another embodiment, useful morphogenic proteins comprise an amino acid sequence encoded by a nucleic acid that hybridizes, under low, medium or high stringency hybridization conditions, to DNA or RNA encoding reference morphogenic protein coding sequences. Exemplary reference sequences include the C-terminal sequences defining the conserved seven-cysteine domains of OP-1, OP-2, BMP-4, BMP-5, BMP-6, 60A, GDF-3, GDF-5, GDF-6, GDF-7, and the like. High stringent hybridization conditions are herein defined as hybridization in 40% formamide, 5×SSPE, 5×Denhardt's Solution, and 0.1% SDS at 37° C. overnight, and washing in 0.1×SSPE, 0.1% SDS at 50° C. Standard stringency conditions are well characterized in commercially available, standard molecular cloning texts. See, for example, Molecular Cloning, A Laboratory Manual, 2nd Ed., ed. by Sambrook et al. (Cold Spring Harbor Laboratory Press 1989); DNA Cloning, Volumes I and II (D. N. Glover ed., 1985); Oligonucleotide Synthesis (M. J. Gait ed., 1984); Nucleic Acid Hybridization (B. D. Hames & S. J. Higgins eds. 1984); and B. Perbal, A Practical Guide To Molecular Cloning (1984).

Suitable in vitro, ex vivo and in vivo bioassays known in the art, including those described herein, may be used to ascertain whether a new BMP-related gene product has a morphogenic activity. Expression and localization studies defining where and when the gene is expressed may also be used to identify potential morphogenic activities. Nucleic acid and protein localization procedures are well known to those of skill in the art (see, e.g., Ausubel et al., eds. Current Protocols in Molecular Cloning, Greene Publishing and Wiley Interscience, New York, 1989).

Many of the identified BMPs are osteogenic and can induce bone and cartilage formation when implanted into mammals. Some BMPs identified based on sequence homology to known osteogenic proteins possess other morphogenic activities such as angiogenic activity and the MPSFs according to this invention may be used to enhance those activities.

That osteogenic proteins originally derived from bone matrix are involved in angiogenesis suggests that these and other members of the BMP family have additional tissue inductive properties that are not yet disclosed. It is envisioned that the MPSFs set forth in this invention can be used to enhance new or known tissue inductive properties of various known morphogenic proteins. It is also envisioned that the invention described herein will be useful for stimulating tissue inductive activities of new morphogenic proteins as they are identified in the future.

Production of Morphogenic Proteins

The morphogenic proteins of this invention can be derived from a variety of sources. For instance, they may be isolated from natural sources, recombinantly produced, or chemically synthesized.

1. Naturally Derived Morphogenic Proteins

The morphogenic proteins used in this invention can be purified from tissue sources, e.g., mammalian tissue sources, using well known techniques. See, e.g., Oppermann et al., U.S. Pat. Nos. 5,324,819 and 5,354,557. If a purification protocol is unpublished, as for a newly identified morphogenic protein, conventional protein purification techniques (e.g., immunoaffinity) may be performed in combination with morphogenic activity assays. Such assays allow the trace of the morphogenic activity through a series of purification steps.

2. Recombinantly Expressed Morphogenic Proteins

In another embodiment of this invention, the morphogenic protein used in this invention is produced by expressing an appropriate recombinant DNA molecule in a host cell. The DNA and amino acid sequences of many BMPs and OPs have been reported, and methods for their recombinant production are published and otherwise known to those of skill in the art. For a general discussion of cloning and recombinant DNA technology, see Ausubel et al., supra; see also Watson et al., Recombinant DNA, 2d ed. 1992 (W. H. Freeman and Co., New York).

The DNA sequences encoding bovine and human BMP-2 (formerly BMP-2A) and BMP-4 (formerly BMP-2B), and processes for recombinantly producing the corresponding proteins are described in U.S. Pat. Nos. 5,011,691, 5,013,649, 5,166,058 and 5,168,050. The DNA and amino acid sequences of human and bovine BMP-5 and BMP-6, and methods for their recombinant production, are disclosed in U.S. Pat. Nos. 5,106,748, and 5,187,076, respectively; see also U.S. Pat. Nos. 5,011,691 and 5,344,654. Methods for OP-1 recombinant expression are disclosed in Oppermann et al., U.S. Pat. Nos. 5,011,691 and 5,258,494. For an alignment of BMP-2, BMP-4, BMP-5, BMP-6 and OP-1 (BMP-7) amino acid sequences, see WO 95/16034. DNA sequences encoding BMP-8 are disclosed in WO 91/18098, and DNA sequences encoding BMP-9 in WO 93/00432. DNA and deduced amino acid sequences encoding BMP-10 and BMP-11 are disclosed in WO 94/26893, and WO 94/26892, respectively. DNA and deduced amino acid sequences for BMP-12 and BMP-13 are disclosed in WO 95/16035. The above patent disclosures, which describe DNA and amino acid sequences, and methods for producing the BMPs and OPs encoded by those sequences, are incorporated herein by reference.

To clone genes that encode new BMPs, OPs and other morphogenic proteins identified in extracts by bioassay, methods entailing “reverse genetics” may be employed. Such methods start with a protein of known or unknown function to obtain the gene that encodes that protein. Standard protein purification techniques may be used as an initial step. If enough protein can be purified to obtain a partial amino acid sequence, a degenerate DNA probe capable of hybridizing to the DNA sequence that encodes that partial amino acid sequence may be designed, synthesized and used as a probe to isolate full-length clones that encode that or a related morphogenic protein.

Alternatively, a partially-purified extract containing the morphogenic protein may be used to raise antibodies directed against that protein. Morphogenic protein-specific antibodies may then be used as a probe to screen expression libraries made from cDNAs (see, e.g., Broome and Gilbert, Proc. Natl. Acad. Sci. U.S.A., 75, pp. 2746-49 (1978); Young and Davis, Proc. Natl. Acad. Sci. U.S.A., 80, pp. 31-35 (1983)).

For cloning and expressing new BMPs, OPs and other morphogenic proteins identified based on DNA sequence homology, the homologous sequences may be cloned and sequenced using standard recombinant DNA techniques. With the DNA sequence available, a DNA fragment encoding the morphogenic protein may be inserted into an expression vector selected to work in conjunction with a desired host expression system. The DNA fragment is cloned into the vector such that its transcription is controlled by a heterologous promoter in the vector, preferably a promoter which may be optionally regulated.

Some host-vector systems appropriate for the recombinant expression of BMPs and OPs are disclosed in the references cited above. Useful host cells include but are not limited to bacteria such as E. coli, yeasts such as Saccharomyces and Picia, insects cells and other primary, transformed or immortalized eukaryotic cultured cells. Preferred eukaryotic host cells include CHO, COS and BSC cells (see below).

An appropriate vector is selected according to the host system selected. Useful vectors include but are not limited to plasmids, cosmids, bacteriophage, insect and animal viral vectors, including those derived from retroviruses and other single and double-stranded DNA viruses.

In one embodiment, the morphogenic protein used in the method of this invention may be derived from a recombinant DNA molecule expressed in a prokaryotic host. Using recombinant DNA techniques, various fusion genes have been constructed to induce recombinant expression of naturally sourced osteogenic sequences in E. coli (see, e.g., Oppermann et al., U.S. Pat. No. 5,354,557, incorporated herein by reference). Using analogous procedures, DNAs comprising truncated forms of naturally sourced morphogenic sequences may be prepared as fusion constructs linked by a sequence coding for the acid labile cleavage site (Asp-Pro) to a leader sequence (such as the “MLE leader”) suitable for promoting expression in E. coli.

In another embodiment, the morphogenic protein used in this invention is expressed using a mammalian host-vector system (e.g., transgenic production or tissue culture production). A morphogenic protein so expressed may resemble more closely the naturally occurring protein. While the glycosylation pattern of the recombinant protein may sometimes differ from that of the natural protein, such differences are often not essential for biological activity of the recombinant protein. Techniques for transfection, expression and purification of recombinant proteins are well known in the art. See, e.g., Ausubel et al., supra, and Bendig, Genetic Engineering, 7, pp. 91-127 (1988).

Mammalian DNA vectors should include appropriate sequences to promote expression of the gene of interest. Such sequences include transcription initiation, termination and enhancer sequences; efficient RNA processing signals such as splicing and polyadenylation signals; mRNA-stabilizing sequences; translation-enhancing sequences (e.g., Kozak consensus sequence); protein-stabilizing sequences; and when desired, sequences that enhance protein secretion.

Restriction maps and sources of various exemplary expression vectors designed for OP-1 expression in mammalian cells have been described in U.S. Pat. No. 5,354,557. Each of these vectors employs a full-length hOP-1 cDNA sequence inserted into the pUC-18 vector. It will be appreciated by those of skill in the art that DNA sequences encoding truncated forms of morphogenic proteins may also be used, provided that the expression vector or host cell provides the sequences necessary to direct processing and secretion of the expressed protein.

Useful promoters include, but are not limited to, the SV40 early and late promoters, the adenovirus major late promoter, the mouse metallothionein-I (“mMT”) promoter, the Rous sarcoma virus (“RSV”) long terminal repeat (“LTR”), the mouse mammary tumor virus (“MMTV”) LTR, and the human cytomegalovirus (“CMV”) major intermediate-early promoter. For instance, a combination of the CMV or MMTV promoter with an enhancer sequence from the RSV LTR has been found to be particularly useful in expressing human osteogenic proteins.

Preferred DNA vectors also include a marker gene (e.g., neomycin or DHFR) and means for amplifying the copy number of the gene of interest. DNA vectors may also comprise stabilizing sequences (e.g., ori- or ARS-like sequences and telomere-like sequences), or may alternatively be designed to favor directed or non-directed integration into the host cell genome.

One method of gene amplification in mammalian cell systems is the use of the selectable dihydrofolate reductase (DHFR) gene in a dhfr- cell line. Generally, the DHFR gene is provided on the vector carrying the gene of interest, and addition of increasing concentrations of the cytotoxic drug methotrexate (MTX) leads to amplification of the DHFR gene copy number, as well as that of the gene physically associated with it. DHFR as a selectable, amplifiable marker gene in transfected Chinese hamster ovary (CHO) cell lines is particularly well characterized in the art. Other useful amplifiable marker genes include the adenosine deaminase (ADA) and glutamine synthetase (GS) genes.

Gene amplification can be further enhanced by modifying marker gene expression regulatory sequences (e.g., enhancer, promoter, and transcription or translation initiation sequences) to reduce the levels of marker protein produced. Lowering the level of DHFR transcription increases the DHFR gene copy number (and the physically-associated gene) to enable the transfected cell to adapt to growth in even low levels of methotrexate (e.g., 0.1 μM MTX). Preferred expression vectors such as pH754 and pH752 (Oppermann et al., U.S. Pat. No. 5,354,557, FIGS. 19C and D) have been manipulated, using standard recombinant DNA technology, to create a weak DHFR promoter. As will be appreciated by those skilled in the art, other useful weak promoters, different from those disclosed herein, can be constructed using standard methods. Other regulatory sequences also can be modified to achieve the same effect.

Another gene amplification scheme relies on the temperature sensitivity (ts) of BSC40-tsA58 cells transfected with an SV40 vector. Temperature reduction to 33° C. stabilizes the temperature-sensitive SV40 T antigen, which leads to the excision and amplification of the integrated transfected vector DNA, thereby amplifying the physically-associated gene of interest.

The choice of cells/cell lines depends on the needs of the skilled practitioner. Monkey kidney cells (COS) provide high levels of transient gene expression and are thus useful for rapidly testing vector construction and the expression of cloned genes. COS cells expressing the gene of interest can be established by transfecting the cells with, e.g., an SV40 vector carrying the gene. Stably transfected cell lines, on the other hand, can be used for long term production of morphogenic proteins. By way of example, both CHO cells and BSC40-tsA58 cells can be used as host cells. Recombinant OP-1 has been expressed in three different cell expression systems: COS cells for rapidly screening the functionality of the various expression constructs, CHO cells for the establishment of stable cell lines, and BSC40-tsA58 cells as an alternative means of producing recombinant OP-1 protein.

Several bone-derived osteogenic proteins (OPs) and BMPs are found as homo- and heterodimers comprising interchain disulfide bonds in their active forms. For instance, BMP-4, BMP-6 and BMP-7 (OP-1)—originally isolated from bone—are bioactive as either homodimers or heterodimers. The ability of OPs and BMPs to form heterodimers may confer additional or altered morphogenic activities on morphogenic proteins. Heterodimers may exhibit qualitatively or quantitatively different binding affinities than homodimers for OP and BMP receptors. Altered binding affinities may in turn result in differential activation of receptors that mediate different signalling pathways, ultimately leading to different biological activities. Altered binding affinities can also be manifested in a tissue or cell type-specific manner, thereby inducing only particular progenitor cell types to undergo proliferation and/or differentiation.

The dimeric proteins can be isolated from the culture media and/or refolded and dimerized in vitro to form biologically active compositions. Heterodimers can be formed in vitro by combining separate, distinct polypeptide chains. Alternatively, heterodimers can be formed in a single cell by co-expressing nucleic acids encoding separate, distinct polypeptide chains. See, e.g., WO 93/09229 and U.S. Pat. No. 5,411,941, for exemplary protocols for heterodimer protein production.

Synthetic Non-Native Morphogenic Proteins

In another embodiment, a morphogenic protein used in the method of this invention may be prepared synthetically. Morphogenic proteins prepared synthetically may be native, or may be non-native proteins, i.e., those not otherwise found in nature.

Non-native morphogenic proteins can be made by mutating native morphogenic proteins. Methods for making mutations that favor refolding and/or assembling subunits into forms that exhibit greater morphogenic activity have been described. See, e.g., U.S. Pat. No. 5,399,677.

Non-native morphogenic proteins can also be synthesized using a series of consensus sequences (U.S. Pat. No. 5,324,819). These consensus sequences were designed based on partial amino acid sequence data obtained from native osteogenic products and on their homologies with other proteins reportedly having a presumed or demonstrated developmental function. Several biosynthetic consensus sequences (called consensus osteogenic proteins or “COPs”) have been expressed as fusion proteins in prokaryotes. Purified fusion proteins may be cleaved, refolded, combined with a hormone and a soluble receptor thereof, implanted in an established animal model and examined for their bone- and/or cartilage-inducing activity. Certain preferred synthetic osteogenic proteins comprise one or both of two synthetic amino acid sequences designated COP5 and COP7.

The amino acid sequences of COP5 and COP7 are shown below, as set forth in Oppermann et al., U.S. Pat. Nos. 5,011,691 and 5,324,819, which are incorporated herein by reference:

| | |
|---|---|
| COP5 | LYVDFS-DVGWDDWIVAPPGYQAFYCHGECPFPLAD |
| COP7 | LYVDFS-DVGWNDWIVAPPGYHAFYCHGECPFPLAD |
| COP5 | HFNSTN--H-AVVQTLVNSVNSKI--PKACCVPTELSA |
| COP7 | HLNSTN--H-AVVQTLVNSVNSKI--PKACCVPTELSA |
| COP5 | ISMLYLDENEKVVLKYNQEMVVEGCGCR |
| COP7 | ISMLYLDENEKVVLKYNQEMVVEGCGCR |

In these amino acid sequences, the dashes (-) are used as fillers only to line up comparable sequences in related proteins. Differences between the aligned amino acid sequences are highlighted.

In one embodiment, the morphogenic protein used in the method of this invention is a synthetic osteogenic protein comprising a partial or complete sequence of a generic sequence described above (SEQ ID NO: 4, 5, 6, 7, or 10) such that it is capable of inducing tissue formation when properly folded and implanted in a mammal. For instance, the synthetic protein can induce bone formation from osteoblasts when implanted in a favorable environment; or it can promote cartilage formation when implanted in an avascular locus or when co-administered with an inhibitor of full bone development.

In another embodiment, the synthetic morphogenic protein used in the method of this invention comprises a sequence sufficiently duplicative of a partial or complete sequence of a COP, e.g., COP5 or COP7. Biosynthetic COP sequences are believed to dimerize during refolding and appear not to be active when reduced. Both homodimeric and heterodimeric COPs are contemplated in this invention. In certain embodiments, this synthetic protein is less than about 200 amino acids long.

These and other synthetic non-native osteogenic proteins may be used in concert with a MPSF and tested using in vitro, ex vivo or in vivo bioassays for progenitor cell induction and tissue regeneration. The proteins in conjunction with the MPSFs of this invention are envisioned to be useful for the repair and regeneration of vascular, bone, cartilage, tendon, ligament, neural and potentially other types of tissue.

Homologous Proteins Having Morphogenic Activity

The morphogenic proteins useful in this invention may be produced by recombinant expression of DNA sequences isolated based on homology with the osteogenic COP consensus sequences described above. Synthetic COP DNA sequences may be used as probes to retrieve related DNA sequences from a variety of species (see, e.g., Oppermann et al., U.S. Pat. Nos. 5,011,591 and 5,258,494, which are incorporated herein by reference).

Morphogenic proteins encoded by a gene that hybridizes with a COP sequence probe are assembled into two subunits disulfide-bonded to produce a heterodimer or homodimer capable of inducing tissue formation when implanted into a mammal. Recombinant BMP-2 and BMP-4 have been shown to have cross-species osteogenic activity as homodimers and as heterodimers assembled with OP-1 subunits.

Morphogenic protein-encoding genes that hybridize to synthetic COP sequence probes include genes encoding Vg1, inhibin, DPP, OP-1, BMP-2 and BMP-4. Vg1 is a known Xenopus laevis morphogenic protein involved in early embryonic patterning. Inhibin is another developmental gene that is a member of the BMP family of proteins from Xenopus laevis. DPP is an amino acid sequence encoded by a Drosophila gene responsible for development of the dorso-ventral pattern. OP-1, BMP-2 and BMP-4 are osteogenic proteins that can induce cartilage, bone and neural tissue formation.

In another embodiment, a morphogenic protein used in the method of this invention may comprise a polypeptide encoded by a nucleic acid that hybridizes under stringent conditions to an “OPS” nucleic acid probe (Oppermann et al., U.S. Pat. No. 5,354,557). “OPS”—standing for OP-1 “short”—refers to the portion of the human OP-1 protein defining the conserved 6 cysteine skeleton in the C-terminal active region (97 amino acids; SEQ ID NO: 2, residues 335-431).

One example of a stringent hybridization condition is hybridization in 4×SSC at 65° C. (or 10° C. higher than the calculated melting temperature for a hybrid between the probe and a nucleic acid sequence containing no mismatched base pairs), followed by washing in 0.1×SSC at the hybridization temperature. Another stringent hybridization condition is hybridization in 50% formamide, 4×SSC at 42° C.

Thus, in view of this disclosure, the skilled practitioner can readily design and synthesize genes, or isolate genes from cDNA or genomic libraries that encode amino acid sequences having morphogenic activity. These genes can be expressed in prokaryotic or eukaryotic host cells to produce large quantities of active osteogenic or otherwise morphogenic proteins. The recombinant proteins may be in native, truncated, mutant, fusion, or other active forms capable of inducing formation of bone, cartilage, or other types of tissue, as demonstrated by in vitro and ex vivo bioassays and in vivo implantation in mammals, including humans.

Morphopenic Protein Stimulatory Factors (MPSF)

A morphogenic protein stimulatory factor (MPSF) used in the method according to this invention is a factor that is capable of stimulating the ability of a morphogenic protein to induce angiogenesis. In one embodiment, the angiogenesis comprises induction of vascular tissue formation from a progenitor cell. In another embodiment of this invention, a method for improving the angiogenic activity of a morphogenic protein in a mammal by coadministering an effective amount of a MPSF is provided. The MPSF may have an additive effect on angiogenesis by the morphogenic protein. Preferably, the MPSF has a synergistic effect on angiogenesis by the morphogenic protein.

The progenitor cell that is induced to proliferate and/or differentiate by the morphogenic protein of this invention is preferably a mammalian cell. Preferred progenitor cells include mammalian endothelial cell progenitor cell, all earlier developmental precursors thereof, and all cells that develop therefrom. However, morphogenic proteins are highly conserved throughout evolution, and non-mammalian progenitor cells are also likely to be stimulated by same- or cross-species morphogenic proteins and MPSF combinations. It is thus envisioned that when schemes become available for implanting xenogeneic cells into humans without causing adverse immunological reactions, non-mammalian progenitor cells stimulated by morphogenic protein and a MPSF according to the procedures set forth herein will be useful for tissue regeneration and repair in humans.

One or more MPSFs are selected for use in concert with one or more morphogenic proteins according to the desired tissue type to be induced and the site at which the morphogenic protein and MPSF will be administered. The particular choice of a morphogenic protein(s)/MPSF(s) combination and the relative concentrations at which they are combined may be varied systematically to optimize the tissue type induced at a selected treatment site using the procedures described herein.

The preferred morphogenic protein stimulatory factors (MPSFs) of this invention are selected from the group consisting of hormones, cytokines and growth factors. In one preferred embodiment, MPSFs for inducing angiogenesis in concert with an osteogenic protein comprise at least one compound selected from the group consisting of fibroblast growth factor (FGF), particularly acidic (aFGF) and basic FGF (bFGF), transforming growth factory-β (TGF-β), transforming growth factor-α (TGF-α), epidermal growth factor (EGF), vascular endothelial growth factor (VEGF), endothelial cell growth factor (ECGF), insulin-like growth factor-1 (IGF-1), hepatocyte growth factor (HGF), platelet activating factor (PAF), interleukin-8 (IL-8), placental growth factor (PGF), proliferin, B61, soluble vascular cell adhesion molecule-1 (SVCAM-1), soluble E-selectin, ephrin, 12-hydorxyeicosatetraenoic acid, tat protein of HIV-1, angiogenin, prostaglandin, particularly PGE2 and amino acid variants thereof. More preferred MPSFs for inducing angiogenesis in concert with an osteogenic protein comprise at least one compound selected from the group consisting of basic fibroblast growth factor (bFGF), platelet derived transforming growth factor-β1 (TGF-β1) and amino acid variants thereof. One most preferred MPSP is basic fibroblast growth factor (bFGF) and amino acid variants thereof. Another most preferred MPSF is platelet derived transforming growth factor-β1 (TGF-β) and amino acid variants thereof.

In another preferred embodiment of this invention, the MPSF comprises a compound or an agent that is capable of increasing the bioactivity of another MPSF. Agents that increase MPSF bioactivity include, for example, those that increase the synthesis, half-life, reactivity with other biomolecules such as binding proteins and receptors, or the bioavailability of the MPSF. These agents may comprise hormones, growth factors, peptides, cytokines, carrier molecules such as proteins or lipids, or other factors that increase the expression or the stability of the MPSF.

For example, when the selected MPSF is FGF, agents that increase its bioactivity include heparan sulfate proteoglycans (HSPGs), which may thus function as MPSFs according to this invention.

Preferably, the MPSF is present in an amount capable of synergistically stimulating the tissue inductive activity of the morphogenic protein in a mammal. The relative concentrations of morphogenic protein and MPSF that will optimally induce tissue formation when administered to a mammal may be determined empirically by the skilled practitioner using the procedures described herein.

Testing Putative Morphogenic Protein Stimulatory Factors To identify a MPSF that is capable of stimulating the angiogenic activity of a chosen morphogenic protein, an appropriate assay is selected. Initially, it is preferable to perform in vitro assays to identify a MPSF that is capable of stimulating the angiogenic activity of a morphogenic protein. A useful in vitro assay is one which monitors a marker known to correlate with the associated differentiation pathway (see Examples 1-3).

Examples 5-6 describe experiments using the osteogenic protein OP-1 to determine its effect on angiogenesis and to identify and optimize an effective concentration of MPSF. OP-1 has some angiogenic activity. Thus, an in vitro assay looking at the expression of an angiogenic-associated marker can be used to identify one or more MPSFs that function in concert with OP-1.

Testing Putative MPSFs Using Angiogenesis Assays

A preferred assay for testing potential MPSFs with OP-1 for angiogenic activity is the chorioallantoic membrane (CAM) assay. The CAM assay is a measure of the angiogenic response. The procedure is generally as follows.

First, a MPSF is identified by picking one or more concentrations of a MPSF and testing them alone or in the presence of a morphogenic protein (Examples 5-6). Second, the amount of MPSF required to achieve optimal, preferably synergistic, tissue induction in concert with the morphogenic protein is determined by generating dose response curves.

Optionally, one or more additional MPSFs that stimulate or otherwise alter the angiogenic activity induced by a morphogenic protein and a first MPSF may be identified and a new multi-factor dose response curve generated.

Utility of Morphogenic Proteins and MPSFs

The morphogenic proteins alone or in combination with MPSFs of this invention will permit the treatment of a variety of injuries or pathologies where vascular tissue formation is required. The morphogenic proteins alone or in combination can ameliorate or remedy the injuries or pathologies by stimulating angiogenesis.

In one embodiment of this invention, a method for inducing angiogenesis in a mammal by administering an effective amount of a morphogenic protein, with the proviso that said morphogenic protein is not BMP-2 or GDF-5 is provided. In another embodiment of this invention a method for improving the angiogenic inductive activity of a morphogenic protein in a mammal by coadministering with the morphogenic protein an effective amount of a morphogenic protein stimulatory factor is provided.

In one preferred embodiment, the morphogenic protein stimulatory factor has synergistic effects on angiogenesis by the morphogenic protein. In another preferred embodiment the morphogenic protein stimulatory factor has additive effects on angiogenesis by the morphogenic protein.

The morphogenic proteins and MPSFs may be administered at the desired locus in a mammal such that the morphogenic proteins and MPSFs are accessible to the appropriate progenitor cells of the mammal. When a combination of morphogenic protein and MPSF is used to induce angiogenesis, they may be administered either simultaneously or separately to a target locus. For example, there may be the morphogenic protein is administered first and then the MPSF is administered. In a preferred embodiment, the target locus is a vascular tissue defect.

### EXAMPLE 1

### Chorioallantoic Membrane (CAM) Assay

Fertile chick eggs (Lowman Brown) were incubated and prepared for bead implantation on the third or fourth day of incubation as described (Vu et al., Lab. Invest., 53, pp. 499-508 (1985); Gould et al., Life Sci., 56, pp. 587-594 (1995), Kirchner et al., Microvasc. Res., 51, pp. 1-14 (1996)). The protein pellets were gently placed on the chorioallantoic membranes (CAMs) on day 10 of incubation. The eggs were then incubated without turning until harvest. On day 15 of incubation, i.e. after a total implantation period of 5 days, the CAMs were fixed in situ with phosphate buffered formalin (10% solution).

### EXAMPLE 2

### Macroscopic Analysis

Within each treatment group, randomly selected CAMs were photographed using a Wild M400 photomacroscope (Wild Heerbrugg Ltd., Switzerland). The CAM photomicrographs were evaluated visually in a masked fashion as previously described (Vu et al., supra; Flamme et al., Development, 111, pp. 683-690 (1991): Olivo et al., Anat. Rec., 234, pp. 105-115 (1992)) with minor modifications. The results were described as: (i) no response: blood vessels undisturbed around the beads and surrounding CAM, (ii) questionable response: blood vessels radiating from the surrounding CAM and directionally shifting towards the beads in a spoke-wheel-like pattern or (iii) positive response: blood vessels converging on the area around the beads in a prominent spoke-wheel pattern.

### EXAMPLE 3

### Microscopic Analysis

The pellets and the adjacent tissue of the CAMs were surgically excised, placed in formalin, dehydrated through ethanol and embedded in paraffin wax as described (Yang and Moses, J. Cell. Biol., 111, pp. 731-341 (1990)). Serial sections of the tissues were cut at 5 μm, mounted on glass slides and stained using a modified Goldner's trichrome technique (Ripamonti et al., Matrix, 12, pp. 369-380 (1992); (Ripamonti et al., Bone Morphogenetic Proteins: Biology, Biochemistry and Reconstructive Surgery, Lindholm T. S. ed., pp. 131-145 (1996); Bradbury and Rae, Bone, In: Theory and Practice of Histological Techniques, Bancroft and Stevens, eds., pp. 113-138 (1996); Page et al., Bone, In: Theory and Practice of Histological Techniques, Bancroft and Stevens, eds., pp. 309-340 (1996)). The stain colors nuclei blue-black, erythrocytes red, cytoplasm red-purple, fibrin red and collagen blue. The sections were examined by light microscopy and photographed using a Provis AX70 research microscope (Olympus Optical Co., Japan). Representative histologic sections were evaluated microscopically with the support of computer software (flexible Image Analysis System® ver. 2.15, CSIR, South Africa) installed in Pentium computer with a color monitor.

The mean CAM thickness (μm) was measured as previously described (Yang and Moses, supra) with minor modification. Briefly, the width of the entire CAM (ecto-, meso- and endoderm jointly) was measured across the central region below the implanted beads and across the peripheral regions distant from the beads using an individual distance array of 5 regularly spaced sampling points. The point intervals were determined with the aid of a superimposed lattice grid (Zeiss Integration Platte II) in order to diminish user-bias. In each representative sample section, the thickness ratio (average thickness of the centrally located regions/average thickness of the peripheral non-reactive regions) was computed. These relative changes in membrane thickness were coupled with the changes in the number, size or density of blood vessels and fibrous tissues in the regions, used for the overall evaluation of the angiogenic responses of the various CAMs.

Based on this qualitative evaluation, the different treatment groups were ranked as (I) weak (negligible or no increase in CAM thickness with limited or no increase in capillaries and fibrous tissues), (ii) moderate (moderate increase in CAM thickness with a moderate increase in capillaries and fibrous tissue), (iii) intense (moderate increase in CAM thickness with extensive increase in capillaries and fibrous tissue) or (iv) very intense (extensive increase in CAM thickness with extensive increase in capillaries and fibrous tissue). The experiments were performed in quadruplicate and repeated at least three times.

### EXAMPLE 4

### Statistical Analysis

Quantifiable data (macroscopic evaluation and thickness ratios) were, respectively, analyzed by Two-way or One-way analysis of variance (ANOVA) using GraphPad Prism™ version 2 (San Diego, USA). Results at p<0.05 were considered significant.

### EXAMPLE 5

### Synergistic Effect of bFGF and TGF-P on OP-1 Induced Angiogenesis—Macroscopic Analysis

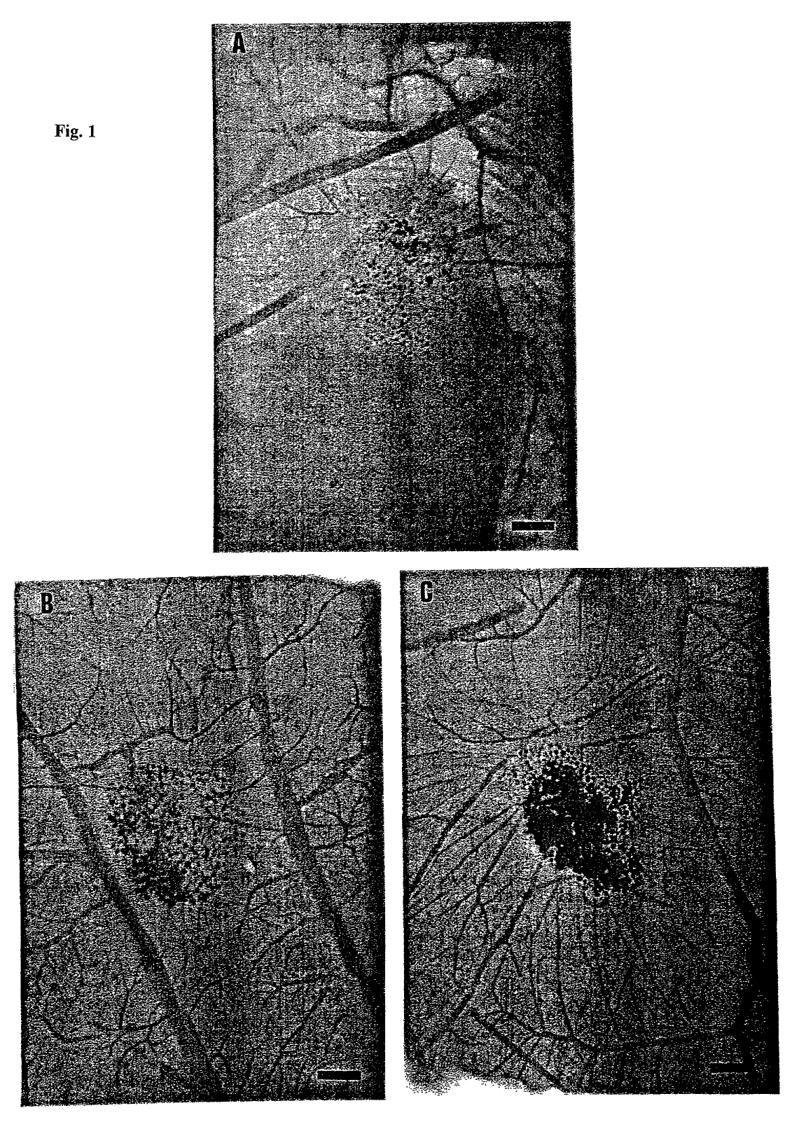

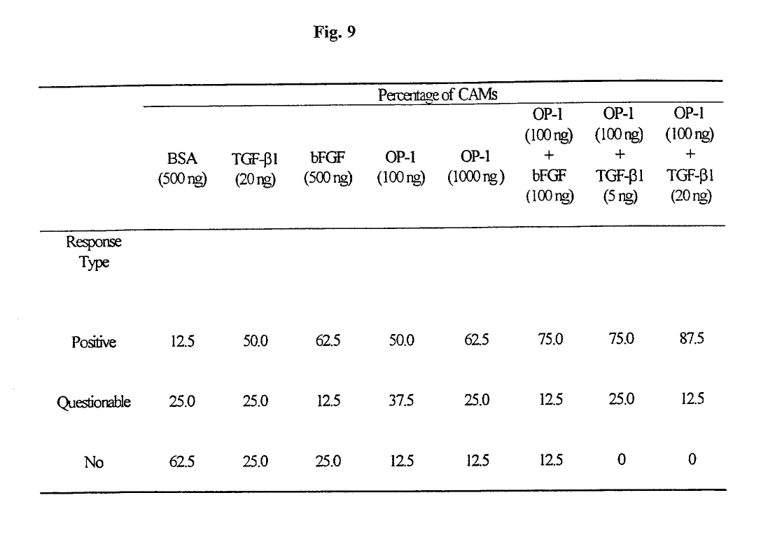

FIGS. 1 and 9 show that the single application of the morphogens pTGF-β1 (20 ng), bFGF (500 ng) or hOP-1 (100 and 1000 ng) and the binary application of hOP-1/bFGF (100/100 ng) or hOP-1/pTGF-β1 (100/5 and 100/20 ng) on the chick chorioallantoic membrane (CAM) demonstrated significantly higher positive angiogenic scores (≧50.0%) compared to the BSA (500 ng) controls (12.5%). The hOP-1/bFGF and hOP-1/pTGF-β1 combinations elicited the highest number of positive responses (≧75%). The highest number of questionable angiogenic responses (37.5%) was produced by the lower dose of hOP-1 (100 ng). The morphogens also exhibited lower non-responsive angiogenic scores(≦25%) compared to the controls (62.5%); with the hOP-1/pTGF-β1 combinations eliciting the loses number of non-responsive scores (0%).

### EXAMPLE 6

### Synergistic Effect of bFGF and TGF-P on OP-1 Induced Angiogenesis—Microscopic Analysis

A. CAM Thickness

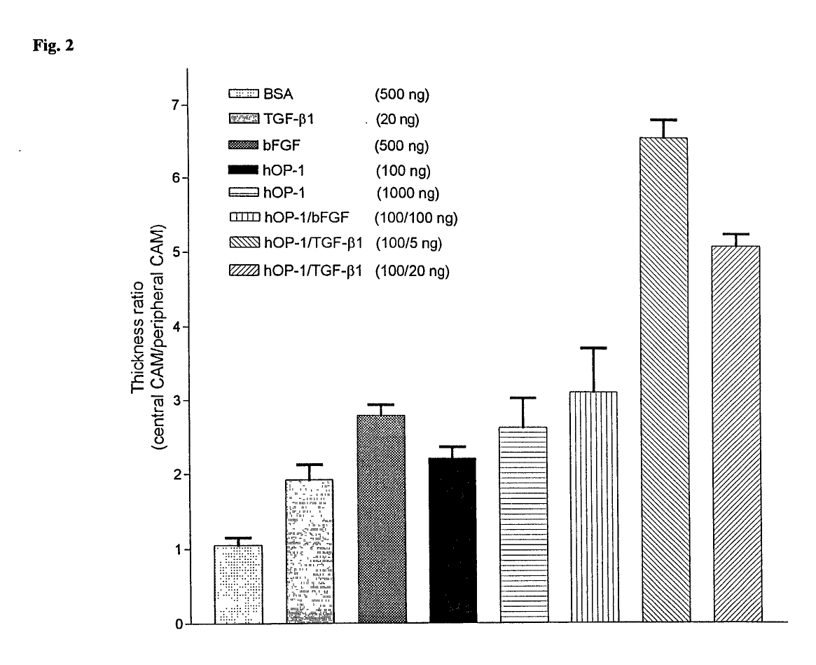

FIGS. 2-8, show that the regions of the CAM in the proximity of the beads soaked in the pTGF-β1 (20 ng), bFGF (500 ng) and hOP-1 (100 and 1000 ng) exhibited a significant increase in the thickness of the CAM compared to the BSA (500 ng) controls. In addition, the binary combination of hOP-1/bFGF (100/100 ng) and hOP-1/pTGF-β1 (100/5 and 100/20 ng) elicited a significantly higher increase in the CAM thickness than the single application of the respective morphogens. the hOP-1/pTGF-β combinations elicited the highest increase in membrane thickness. All the increases in the thickness of the reactive CAMS were accompanied by significant changes in the cell morphology, including an increase in the number and size of blood vessels with nucleated erythrocytes and an increase in fibrous tissue density (fibroplasia).

B. Overall Angiogenic Score

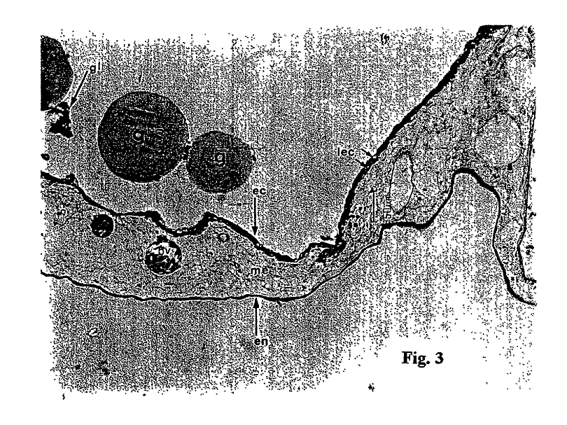

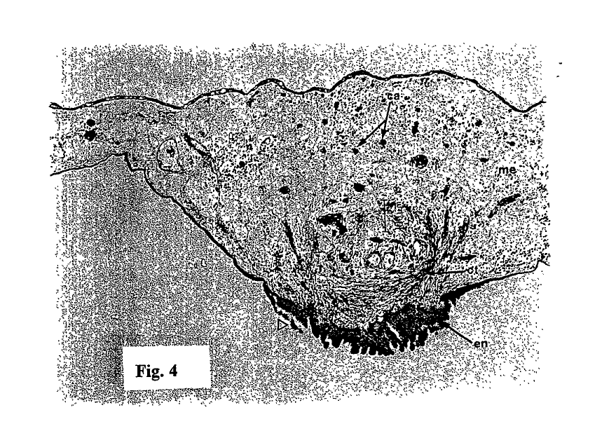

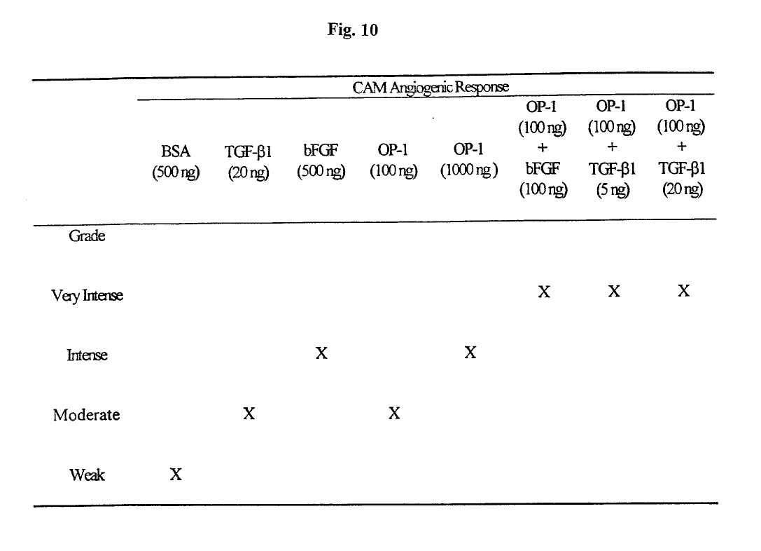

Control: beads soaked with 500 ng BSA resulted in a negligible change in the overall thickness of the CAM (FIG. 2) and a weak or negligible overall angiogenic reaction in the CAM (FIG. 10). As shown in FIG. 3, the ectoderm, mesoderm and endoderm of the CAM beneath the beads developed in a virtually normal pattern when compared to the adjacent non-exposed CAM. The ectoderm and endoderm were flat, single-layered or simple epithelia in the entire expanse of the CAM. The ectoderm, showed normal development of the intradermal capillaries. The mesoderm showed mainly sparsely arranged fibrous tissue with scattered blood vessels with nucleated erythrocytes localized centrally and also adjacent to the ectoderm. The mesoderm adjacent to the endoderm was deficient of blood vessels. pTGF-β1: The application of 20 ng pTGF-β1 resulted in a moderate increase in the thickness of the reactive CAM (FIG. 2) and a moderate overall angiogenic response (FIG. 10). FIG. 4 shows that the reaction center was primarily located in the region of the mesoderm adjacent to the endoderm. There was very marked expansion or thickening of the mesoderm and very intense stratification of the endoderm, with signs of shedding of the outermost cell layers of the stratified epithelium. The mesoderm was also characterized by a widespread increase in the number of capillary blood vessels, as well as increases in the density of the mesenchymal stroma through a condensation of fibroblasts and connective tissue fibers, including blue-staining collagen fibers, adjacent to the endoderm. The ectoderm, in some sections, was altered into a bilayered squamous epithelium.

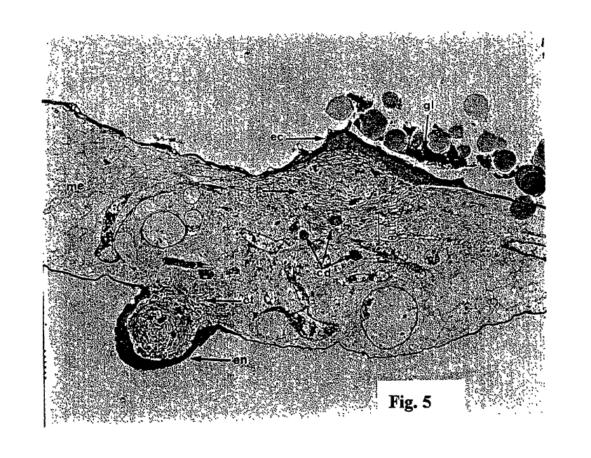

bFGF: The application of 500 ng bFGF resulted in a moderate increase in the thickness of the reactive CAM (FIG. 2) and an intense overall angiogenic response (FIG. 10). The histological features of the reaction show that the response was characterized by intense stratification of both ectoderm and endoderm (FIG. 5). The expanded mesoderm was characterized by augmentation of large capillary blood vessels and an increase in the density of new capillaries and fibrous tissue most primarily in the regions adjacent to both the ectoderm and the endoderm. Blue staining collagen fibers were distributed widely in the reactive mesoderm. Clusters of cells, with a similar morphological appearance to and, presumably, contiguous with the stratified ectoderm were observed in the mesoderm.

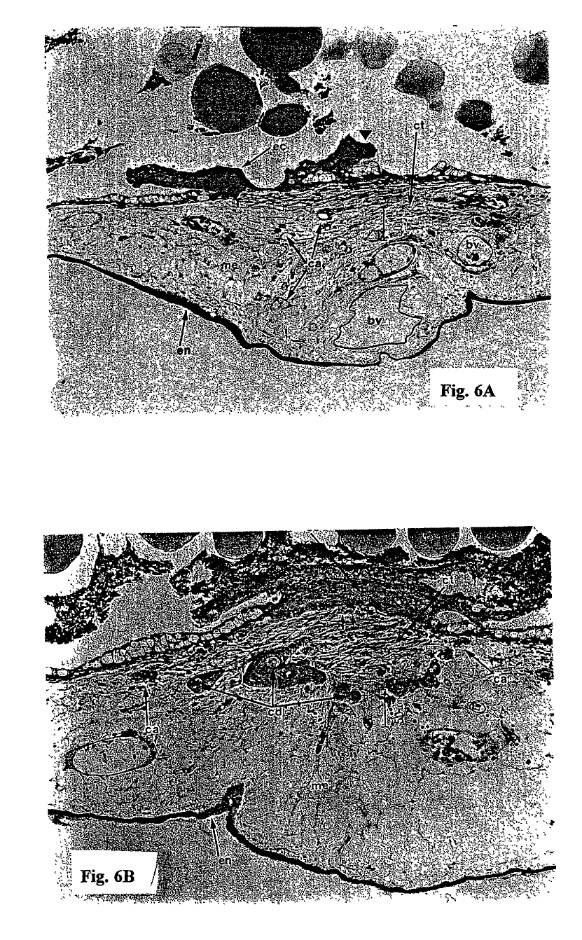

hOP-1: The application of 100 ng and 1000 ng of hOP-1 resulted in a dose-dependent moderate to high increases in the thickness of the reactive CAM (FIG. 2) and moderate to intense overall angiogenic responses, respectively (FIG. 10). The reaction of the CAM to 100 ng hOP-1 (FIG. 6A) was primarily localized at the region of the mesoderm subadjacent to the ectoderm. There was intense stratification of the ectoderm and a weak growth of the endoderm. The mesoderm was expanded, with numerous capillaries and diffuse fibrous tissue distributed mainly in the region near the ectoderm. The reaction to 1000 ng of hOP-1 (FIG. 6B) was also mainly confined to the region of the mesoderm subadjacent to the ectoderm but was more intense than the response elicited by 100 ng of hOP-1. There was very intense stratification of the ectoderm and a moderate cellular expansion of the endoderm. The mesoderm was enlarged, with new capillaries and very dense fibrous tissue distributed mainly in the region subadjacent to the ectoderm. The previously intraectodermal capillaries were located underneath the blood vessel-free stratified ectoderm. In the mesoderm, hydropic cells and necrotic cells were observed in a few groups of cells displaying a morphological appearance identical to the cells of the stratified ectoderm. With both doses of hOP-1 (FIGS. 6A and 6B) there were multiple distended blood vessels with nucleated erythrocytes in the mesoderm.

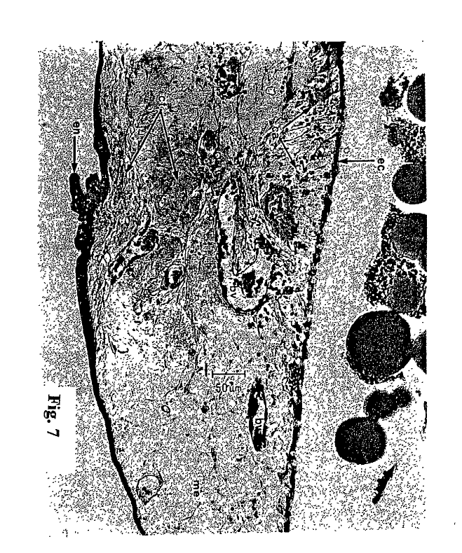

hOP-1/bFGF: The binary application of 100 ng hOP-1 with 100 ng bFGF resulted in a moderate to high increase in the thickness of the reactive CAM (FIG. 2) and a very intense angiogenic response (FIG. 10). The combination resulted in intense alteration of the ectoderm, mesoderm, and endoderm (FIG. 7). The ectodermal epithelium was thickened via stratification and the endodermal cells acquired a columnar shape in addition to cellular hypertrophy. The mesoderm was more consolidated, exhibiting an increased density of fibroblasts and small blood vessels which were widely distributed throughout the reactive region of the CAM. The fibrous tissue, comprising mainly blue-staining collagen, was very dense and spread throughout the perimeter of the reactive mesoderm.

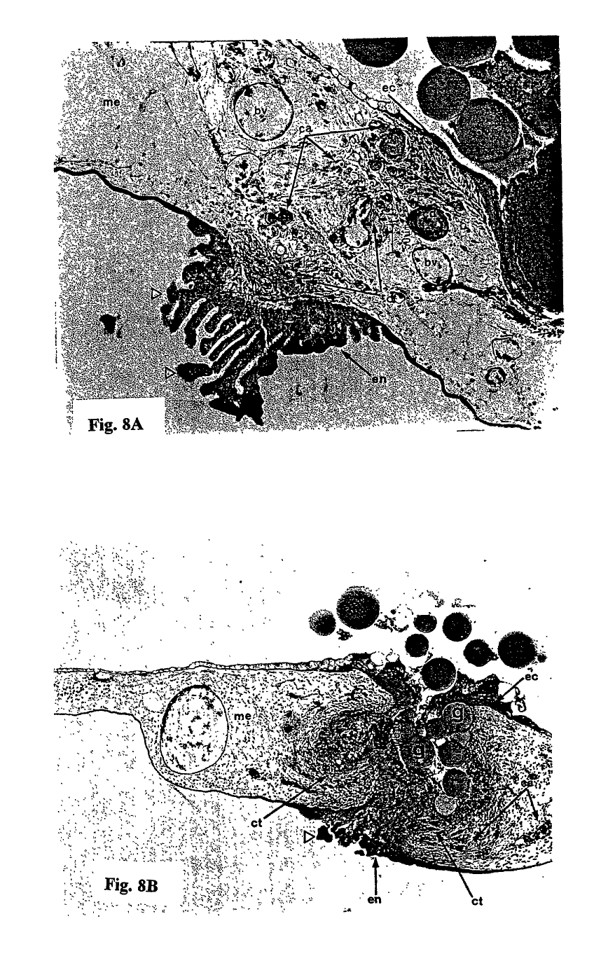

hOP-1/pTGF-β1: The binary application of 100 ng hOP-1 with pTGF-β1 (5 and 20 ng) exhibited a very high increase in the thickness of the reactive CAM (FIG. 2) and a very intense overall angiogenic response (FIG. 10). The increase in the CAM thickness was highest among all the applied morphogenic proteins and MPSFs. All the three layers of the CAM were characterized by very intense hyperplasia (FIGS. 8A and 8B). The responses resulting from both applications were characterized by a high condensation of mesenchyme and fibrous tissue accompanying and extensive proliferation of large and small blood vessels. There was also the presence of dead cells in the mesoderm that were located within groups of cells morphologically identical to the cells of the stratified ectoderm. A concomitant envelopment of the gel beads by the CAM tissue was frequently evident (FIG. 8B).

While we have described a number of embodiments of this invention, it is apparent that our basic constructions may be altered to provide other embodiments which utilize the methods of this invention. Therefore, it will be appreciated that the scope of this invention is to be defined by the appended claims, rather than by the specific embodiments which have been presented by way of example.

What is claimed:

1. A method for inducing angiogenesis in a mammal by administering an effective amount of a morphogenic protein; with the proviso that said morphogenic protein is not BMP-2 or GDF-5.

2. A method for improving the angiogenic inductive activity of a morphogenic protein in a mammal by coadministering with the morphogenic protein an effective amount of a morphogenic protein stimulatory factor.

3. The method according to claim 2, wherein the morphogenic protein stimulatory factor has additive effects on angiogenesis by the morphogenic protein.

4. The method according to claim 2, wherein the morphogenic protein stimulatory factor has synergistic effects on angiogenesis by the morphogenic protein.

5. The method according to any one of claims 1 to 4, wherein the morphogenic protein is an osteogenic protein that is capable of inducing angiogenesis.

6. The method according to any one of claims 1 to 4, wherein the morphogenic protein comprises an amino acid sequence selected from the group consisting of BMP-3, BMP-4, BMP-5, BMP-6, OP-1 (BMP-7), BMP-8, BMP-9, BMP-10, BMP-11, BMP-12, BMP-13, BMP-14, BMP-15, COP-5, COP-7 and an amino acid sequence variant thereof.

7. The method according to any one of claims 1 to 4, wherein the morphogenic protein is a monomeric species.

8. The method according to claim 7, wherein the monomeric species is selected from the group consisting of OP-1, BMP-5, BMP-6, BMP-8, GDF-6, GDF-7 and amino acid sequence variants thereof.

9. The method according to any one of claims 1 to 4, wherein the morphogenic protein comprises a disulfide bonded dimeric species.

10. The method according to claim 9, wherein the dimeric species comprises a polypeptide selected from the group consisting of OP-1, BMP-5, BMP-6, BMP-8, GDF-6, GDF-7 and amino acid sequence variants thereof.

11. The method according to any one of claims 1 to 4, wherein the morphogenic protein is OP-1.

12. The method according to any one of claims 1 to 4, wherein the morphogenic protein is produced by the expression of a recombinant DNA molecule in a host cell.

13. The method according to any one of claims 2 to 4, wherein the morphogenic protein stimulatory factor comprises at least one compound selected from the group consisting acidic fibroblast growth factor (aFGF), basic fibroblast growth factor FGF (bFGF), transforming growth factor-β (TGF-β), transforming growth factor-α (TGF-α), epidermal growth factor (EGF), vascular endothelial growth factor (VEGF), endothelial cell growth factor (ECGF), insulin-like growth factor-1 (IGF-1), hepatocyte growth factor (HGF), platelet activating factor (PAF), interleukin-8 (IL-8), placental growth factor (PGF), proliferin, B61, soluble vascular cell adhesion molecule-1 (SVCAM-1), soluble E-selectin, ephrin, 12-hydorxyeicosatetraenoic acid, tat protein of HIV-1, angiogenin, prostaglandin and amino acid sequence variants thereof.

14. The method according to any one of claims 2 to 4, wherein the morphogenic protein stimulatory factor comprises at least one compound selected from the group consisting of basic fibroblast growth factor (bFGF), platelet derived transforming growth factor-β1 (TGF-β1) and amino acid sequence variants thereof.

15. The method according to any one of claims 2 to 4, wherein the morphogenic protein stimulatory factor is selected from the group consisting of basic fibroblast growth factor (bFGF) and amino acid sequence variants thereof.

16. The method according to any one of claims 2 to 4, wherein the morphogenic protein stimulatory factor is selected from the group consisting of platelet derived transforming growth factor-β1 (TGF-β1) and amino acid sequence variants thereof.

17. The method according to any one of claims 2 to 4, wherein the morphogenic protein and the morphogenic protein stimulatory factor are administered simultaneously to a target locus.

18. The method according to any one of claims 2 to 4, wherein the morphogenic protein and the morphogenic protein stimulatory factor are administered separately to a target locus.

19. The method according to claim 17, wherein the target locus is a vascular tissue defect.

20. The method according to claim 18, wherein the target locus is a vascular tissue defect.


SEQUENCE LISTING (10 sequences)

SEQ ID NO: 1 - DNA, 1822 bp, Homo sapiens
  CDS (49)..(1341)
ggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaaaaaaaaaaaaaaaa

SEQ ID NO: 2 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 3 - protein, 102 aa, Artificial Sequence
  Description of Artificial Sequence OPX
CXXHELYVSFXDLGWXDWXIAPXGYXAYYCEGECXFPLXSXMNATNHAIXQXLVHXXXPXXVPKXCCAPTXLXAXSVLYXDXSXNVILXKXRNMVVXACGCH

SEQ ID NO: 4 - protein, 97 aa, Artificial Sequence
  Description of Artificial Sequence Generic-Seq-7
LXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 5 - protein, 102 aa, Artificial Sequence
  Description of Artificial Sequence Generic-Seq-8
CXXXXLXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 6 - protein, 97 aa, Artificial Sequence
  Description of Artificial Sequence Generic-Seq-9
XXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 7 - protein, 102 aa, Artificial Sequence
  Description of Artificial Sequence Generic-Seq-10
CXXXXXXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 8 - protein, 5 aa, Artificial Sequence
  Description of Artificial Sequence Generic Sequence
CXXXX

SEQ ID NO: 9 - protein, 5 aa, Artificial Sequence
  Description of Artificial Sequence Generic Sequence
CXXXX

SEQ ID NO: 10 - protein, 102 aa, Artificial Sequence
  Description of Artificial Sequence Generic Sequence
CXXXXLXVXFXDXEWXXWXXXPXGXXAXYCXGXCXXPXXXXXXXXNHAXXQXXVXXXNXXXXPXXCCXPXXXXXXXXLXXXXXXXVXLXXYXXMXVXXCXCX